(19) United States
(12) Patent Application Publication
(10) Pub. No.: US 2026/0115449 A1
(43) Pub. Date: Apr. 30, 2026

(54) INTRAVASCULAR BLOOD PUMPS AND METHODS OF USE

(71) Applicant: Shifamed Holdings, LLC, Campbell, CA (US)

(72) Inventors: Daniel HILDEBRAND, Santa Cruz, CA (US); Michael CALOMENI, San Jose, CA (US); Brian D. BRANDT, Morgan Hill, CA (US); Janine ROBINSON, Half Moon Bay, CA (US); Ari RYAN, San Jose, CA (US); Daniel VARGHAI, Scotts Valley, CA (US); Reza SHIRAZI, San Jose, CA (US); Seyed Mostafa GHOREYSHI, Campbell, CA (US); Amr SALAHIEH, Saratoga, CA (US)

(21) Appl. No.: 19/006,983

(22) Filed: Dec. 31, 2024

Related U.S. Application Data
(62) Division of application No. 16/595,280, filed on Oct. 7, 2019, now Pat. No. 12,220,570.
(60) Provisional application No. 62/741,970, filed on Oct. 5, 2018.
(60) Provisional application No. 62/778,804, filed on Dec. 12, 2018.
(60) Provisional application No. 62/905,818, filed on Sep. 25, 2019.

(51) Int. Cl. A61M 60/411; A61M 60/13; A61M 60/216; A61M 60/414; A61M 60/422; A61M 60/808; A61M 60/812; A61M 60/818; A61M 60/831

(52) U.S. Cl. CPC A61M 60/411; A61M 60/13; A61M 60/216; A61M 60/422; A61M 60/808; A61M 60/812; A61M 60/818; A61M 60/831; A61M 60/414

(57) ABSTRACT

Intravascular blood pumps and methods of use. The blood pump include a pump portion that includes a collapsible blood conduit defining a blood flow lumen between an inflow and an outflow. The pump portion includes a distal collapsible impeller axially spaced from a proximal collapsible impeller, at least a portion of each of the distal and proximal collapsible impellers disposed between the inflow and the outflow.

### CROSS REFERENCE TO RELATED APPLICATIONS

This application is a divisional of U.S. Application No. 16/595,280, filed Oct. 7, 2019, which claims the benefit of the following U.S. Provisional Applications, each of which is incorporated herein by reference in its entirety for all purposes: App. No. 62/741,970, filed Oct. 5, 2018, App. No. 62/778,804, filed Dec. 12, 2018, and App. No. 62/905,818, filed Sep. 25, 2019.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. There is a need for ways to improve the volume or stability of cardiac outflow for these patients, especially during corrective procedures.

Intra-aortic balloon pumps (IABP) are commonly used to support circulatory function, such as treating heart failure patients. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

An IABP commonly works by being placed within the aorta and being inflated and deflated in counterpulsation fashion with the heart contractions, and one of the functions is to attempt to provide additive support to the circulatory system.

More recently, minimally-invasive rotary blood pumps have been developed that can be inserted into the body in connection with the cardiovascular system, such as pumping arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient.

The smallest rotary blood pumps currently available can be percutaneously inserted into the vasculature of a patient through an access sheath, thereby not requiring surgical intervention, or through a vascular access graft. A description of this type of device is a percutaneously-inserted ventricular support device.

There is a need to provide additional improvements to the field of ventricular support devices and similar blood pumps for treating compromised cardiac blood flow.

### SUMMARY OF THE DISCLOSURE

The present disclosure relates to fluid movement devices, such as intravascular blood pumps, and their methods of use.

One aspect of the disclosure is an intravascular blood pump, comprising a pump portion that includes a collapsible blood conduit defining a blood flow lumen between an inflow and an outflow; a distal collapsible impeller axially spaced from a proximal collapsible impeller, at least a portion of each of the distal and proximal collapsible impellers disposed between the inflow and the outflow. The blood pump may include one or more stators positioned in the blood flow lumen when the pump is expanded, the stators axially between the distal and proximal impellers. Any of the one or more stators can include one or more blood flow modifiers or blood flow modifying elements (e.g., blades) disposed axially between the distal and collapsible impellers, the blood flow modifiers having at least one surface configured to modify or influence the flow of blood between the impellers.

In this aspect, any of the flow modifiers may be considered, alone or together, a stator, configured to increase pressure between first and second impellers. There may be multiple stators, axially spaced apart, wherein each of the stators may be considered to have a plurality of flow modifying elements (which may also be referred to as flow modifiers or a derivative thereof).

In this aspect, any blood flow modifier of any stator may be disposed between the shroud and a longitudinal axis of the pump portion (even if the pump portion includes a bend formed therein). Any blood flow modifier may be disposed between the shroud and a central tubular element (e.g., a hub), such as a tubular shaft not adapted to rotate with the impellers, and/or a tubular element adapted to rotate with the impellers.

In this aspect, the one or more blood flow modifiers may be secured to a surface of the collapsible blood conduit.

In this aspect, the one or more blood flow modifiers may extend radially inward from a surface of the collapsible blood conduit, each of the one or more blood flow modifiers having at least one axially extending surface configured to modify the flow of blood between the distal and proximal impellers.

In this aspect, the one or more blood flow modifiers may extend radially outward from a central hub, and may or may not contact the blood conduit.

In this aspect, the one or more blood flow modifiers may extend radially outward from a central hub, and may or may not be secured to the blood conduit.

In this aspect, the one or more blood flow modifiers may extend radially inward from a surface of the collapsible blood conduit, and may or may not extend to a central hub.

In this aspect, the one or more blood flow modifiers may not extend radially to a central hub.

In this aspect, a radially innermost section of each of the one or more blood flow modifiers may be a free section.

In this aspect, the one or more flow modifiers may be integrally formed with at least a portion of the collapsible blood conduit. The one or more flow modifiers may be integral to a scaffold of the collapsible blood conduit. The one or more flow modifiers may be biased to a deployed configuration in which they extend radially inward relative to an outer section of the scaffold.

In this aspect, the one or more blood flow modifiers may extend radially to a central hub.

In this aspect, the one or more blood flow modifiers may be secured to and extend radially inward from an outer annular member that does not extend axially all the way from the inflow to the outflow. The outer annular member may provide radial support to the collapsible blood conduit.

In this aspect, the one or more blood flow modifiers may be formed of polymeric material.

In this aspect, the one or more blood flow modifiers may have radially outer ends that have at least one surface with a configuration that is shaped to stably interface with a corresponding portion of the collapsible blood conduit. The blood pump may further comprise a scaffold having one or more blood flow modifier apertures therethrough, each of the radially outer ends having a configuration shaped to stably interface with one of the blood flow modifier apertures. The pump can further include a membrane layer extending over the scaffold that helps secure the one or more blood flow modifiers to the apertures. The apertures may axially extend and be parallel to a long axis of the scaffold. The pump may further include a self-expanding scaffold, the one or more blood flow modifiers having radially outer ends that have a configuration shaped to stably interface with the self-expanding scaffold. The one or more blood flow modifiers may be made of a different material (e.g., a polymeric material) than the scaffold material, and may be more flexible than the scaffold material.

In this aspect the one or more blood flow modifiers may comprise at least four blood flow modifiers.

In this aspect, the one or more blood flow modifiers may each be secured to one of one or more struts, wherein the struts define a portion of an expandable basket in which the proximal impeller or the distal impeller is disposed. The pump portion may further comprise a membrane layer secured to (directly or indirectly) to the expandable basket, the membrane layer at least partially defining the blood conduit. The one or more struts may be proximal struts of the expandable basket, which may be a distal basket or a proximal basket. The struts may be at a non-orthogonal angle relative to a long axis of the pump portion at the location of the strut.

In this aspect, the one or more blood flow modifiers may have an inner free end that is disposed parallel to a longitudinal axis of the pump portion where the flow modifier is disposed. The collapsible blood conduit may include one or more bends formed along its length, the one or more bends axially spaced from the one or more blood flow modifiers.

In this aspect the one or more blood flow modifiers may be integrally formed with at least one other component of the collapsible blood conduit.

In this aspect, the one or more blood flow modifiers may have radially outer sections that are secured to and extend from the collapsible blood conduit along a length of at least 1 mm and not more than 15 cm, optionally along a length of at least 1 mm and not more than 10 cm, optionally not more than 9 cm, not more than 8 cm, not more than 7 cm, not more than 6 cm, or not more than 5 cm.

In this aspect, the one or more blood flow modifiers may have radially outer sections that are secured to and extend from the blood conduit that are longer than radially inner edges of the blood flow modifier.

In this aspect, the one or more blood flow modifiers have a distal end surface and a proximal end surface, at least one of the ends being tapered.

In this aspect, the pump portion may comprise a membrane that helps secure the one or more blood flow modifiers to the blood conduit.

In this aspect, the one or more blood flow modifiers may be self-deploying.

In this aspect, the at least one axially extending surface may be configured to transition the blood flow towards laminar flow.

In this aspect, the one or more blood flow modifiers may be collapsible between an expanded configuration and a collapsed configuration.

In this aspect, the one or more blood flow modifiers may be at least one of moveable or reconfigurable between a first position and a deployed position.

In this aspect, the one or more blood flow modifiers are positioned closely next to at least one of the proximal impeller and the distal impeller when the proximal and distal impellers and in expanded configurations.

In this aspect, the one or more blood flow modifiers may be closer to the proximal impeller than to the distal impeller.

In this aspect, the one or more blood flow modifiers may be closer to the distal impeller than to the proximal impeller.

In this aspect, a first end of the one or more blood flow modifiers may be 0.01mm-20 mm from at least one of the distal and proximal impellers.

In this aspect, the one or more blood flow modifiers may be secured to (optionally integral with) an annular member that provides radially support for one or more of an impeller basket or scaffold of the blood conduit.

In this aspect, the one or more blood flow modifiers may be part of a collapsible intermediate member positioned and adapted to provide radial support to the blood conduit.

In this aspect, the one or more blood flow modifiers may be part of a collapsible intermediate member positioned to maintain tip gap between at least one of the impellers and the blood conduit.

In this aspect, a distal region of the one or more stators may be configured to act as a diffuser to fluid in the fluid conduit to recover pressure from the distal impeller, and wherein a proximal region of the one or more fluid modifiers may be configured to act as a stator to direct flow towards the proximal impeller.

One aspect of the disclosure is an intravascular blood pump, comprising: a pump portion that includes: a collapsible blood conduit defining a blood flow lumen between an inflow and an outflow; a distal collapsible impeller axially spaced from a proximal collapsible impeller, at least a portion of each of the distal and proximal collapsible impellers disposed between the inflow and the outflow; and one or more stators, each including one or more blood flow modifiers disposed axially between the distal and collapsible impellers, each of the one or more blood flow modifiers having at least one axially extending surface configured to increase fluid pressure between the distal impeller and the proximal impeller.

In this aspect, the at least one axially extending surface may be configured to transition the flow towards laminar flow.

In this aspect, the one or more blood flow modifiers may be secured to and extending radially inward from a surface of the collapsible blood conduit.

In this aspect, the one or more flow modifiers include any feature of any of the flow modifying elements herein.

One aspect of the disclosure is an intravascular blood pump, comprising: a pump portion that includes: a collapsible blood conduit defining a blood flow lumen between an inflow and an outflow; a proximal collapsible impeller axially spaced from a distal collapsible impeller, at least a portion of each of the distal and proximal collapsible impellers disposed between the inflow and the outflow; and a proximal collapsible basket in which the proximal impeller is disposed, the proximal collapsible basket providing radial support to the blood conduit at the location of the proximal impeller; a distal collapsible basket in which the distal impeller is disposed, the distal collapsible basket providing radial support to the blood conduit at the location of the distal impeller; a collapsible radial support member supporting one or more of a distal region of the proximal collapsible basket, a proximal region of the distal collapsible basket, or a central section of the blood conduit disposed axially between the proximal basket and the distal basket.

In this aspect, the radial support member may include an annular peripheral member and a plurality of support elements extending radially inward from the annular peripheral member. The plurality of support elements may or may not extend to a central hub. The plurality of support elements may have radially inner free ends.

In this aspect, the radial support member may support the distal region of the proximal basket.

This aspect may further include a second radial support member spaced axially from the radial support member, the second radial support member can be positioned to radially support the proximal region of the distal basket. A second radial support member may include a second annular peripheral member and a plurality of second support elements extending radially inward from the second annular peripheral member.

In this aspect, the radial support member may support the proximal region of the distal basket.

In this aspect, the collapsible radial support member may comprise a stator including one or more blood modifying elements, such as any of the blood modifying elements herein.

One aspect of this disclosure is an intravascular blood pump, comprising: a pump portion that includes: a collapsible blood conduit defining a blood flow lumen between an inflow and an outflow; a proximal collapsible impeller axially spaced from a distal collapsible impeller, at least a portion of each of the distal and proximal collapsible impellers disposed between the inflow and the outflow; and a proximal collapsible basket in which the proximal impeller is disposed, the proximal collapsible basket providing radial support to the blood conduit at the location of the proximal impeller; a distal collapsible basket in which the distal impeller is disposed, the distal collapsible basket providing radial support to the blood conduit at the location of the distal impeller; a collapsible radial support member including an annular peripheral member and a plurality of support elements extending radially inward from the annular peripheral member, the collapsible radial support radially supporting one or more of a distal region of the proximal collapsible basket, a proximal region of the distal collapsible basket, or a central section of the blood conduit disposed axially between the proximal basket and the distal basket.

In this aspect, the plurality of support elements may or may not extend to a central hub.

In this aspect, the plurality of support elements may have radially inner free ends.

In this aspect, the collapsible radial support may be disposed radially within at least one of the distal region of the proximal collapsible basket, a proximal region of the distal collapsible basket

In this aspect, the collapsible radial support may be disposed radially within the distal region of the proximal basket, wherein the proximal basket includes a plurality of proximal struts but does not include a plurality of distal struts.

In this aspect, the collapsible radial support may comprise a stator, the stator including the plurality of support elements. The plurality of support elements can be configured to increase fluid pressure between the distal and proximal impellers.

One aspect of the disclosure is a repositionable blood pump, comprising: an elongate member including a pump portion, the elongate member sized for intravascular positioning in a subject; a proximal portion from which the elongate member extends distally, the proximal portion sized to be maintained outside of the subject, the proximal portion including a motor assembly coupling region that is configured to securely interface with a motor assembly, the proximal portion including a proximal guidewire port, the guidewire port positioned relative to the motor assembly coupling region such that the guidewire port is accessible for a guidewire to be advanced into the port while the motor assembly is securely interfaced with the motor coupling region.

In this aspect, the proximal region may include a rotatable member in rotational communication with an impeller in the blood pump, wherein the motor assembly coupling region and the motor assembly are configured such that when the motor assembly is securely interfaced with the motor coupling region, the motor assembly is in rotational communication with the rotatable member. The rotatable member may have a portion of a guidewire path formed therein, and wherein when the rotatable member is in a rotationally aligned position, the guidewire path extends from the portion of the guidewire path in the rotatable member to the guidewire port, and when the rotatable member is in a rotationally misaligned position, the guidewire path does not extend from the portion of the guidewire path in the rotatable member to the guidewire port. The portion of the guidewire path formed in the rotatable member may be at least partially curved, and optionally has a proximal port in the rotatable member in a radially side surface of the rotatable member.

In this aspect, the guidewire port may be in a side surface of the proximal portion.

In this aspect, the guidewire port may be configured to securely interface with a fluid line coupler.

This aspect may further comprise any suitable feature or element described herein.

One aspect of the disclosure is a method of using an intravascular blood pump, comprising: activating a motor to cause rotation of an impeller while a guidewire port is outside of a patient and not covered by the motor assembly. This aspect may further comprise any suitable method step herein.

One aspect of the disclosure is a method of using an intravascular blood pump, comprising: while a motor assembly is secured to a proximal portion of an intravascular blood pump apparatus, inserting a guidewire into a guidewire port disposed in a proximal portion of an intravascular blood pump when the proximal portion is disposed external to a subject.

In this aspect, the guidewire port may be in a radially side surface of the proximal portion, and inserting the guidewire may comprise inserting the guidewire into the guidewire port in the radially side surface.

This aspect may also include, while the motor assembly is secured to the proximal portion, repositioning a pump portion of the intravascular blood pump.

This aspect may also include, after the insertion step, removing the guidewire from the guidewire port while the motor assembly is secured to a proximal portion of an intravascular blood pump apparatus.

This aspect may also include any other suitable method step herein.

One aspect of the disclosure is a method of using an intravascular blood pump, comprising: while a motor assembly is secured to a proximal portion of an intravascular blood pump apparatus, removing a guidewire through a guidewire port disposed in a proximal portion of an intravascular blood pump when the proximal portion is disposed external to a subject.

In this aspect, the guidewire port may be in a radially side surface of the proximal portion, and wherein removing the guidewire may comprise removing the guidewire from the guidewire port in the radially side surface.

This aspect may further comprise either receiving outlet fluid from the proximal guidewire port into a fluid line in fluid communication with the guidewire lumen, or advancing inlet fluid into the guidewire port from a fluid line.

One aspect of the disclosure is an intravascular blood pump, comprising: an elongate member including a blood pump, the elongate member sized for intravascular positioning in a subject; a proximal portion from which the elongate member extends distally, the proximal portion sized to be maintained outside of the subject and including a guidewire port extending through a radially side surface of the proximal portion. This aspect may further include any other suitable feature or element described herein.

One aspect of the disclosure is an intravascular blood pump, comprising: an elongate member including a blood pump, the elongate member sized for intravascular positioning in a subject; a proximal portion from which the elongate member extends distally, the proximal portion sized to be maintained outside of the subject, the proximal portion including a portion of a guidewire pathway with a bend formed therein.

In this aspect, the proximal portion may include a rotatable component in rotational communication with an impeller in a pump portion of the elongate member, the rotatable component may have the portion of the guidewire pathway with the bend formed therein.

In this aspect, the proximal portion may further comprise a guidewire port formed in a radially side surface of the proximal portion.

In this aspect, the guidewire path may include the guidewire port and the portion of the guidewire path with the bend when the rotatable component is a rotationally aligned position.

This aspect may further include any other suitable feature or element described herein.

One aspect of the disclosure is an intravascular blood pump, comprising: an elongate member including a blood pump, the elongate member sized for intravascular positioning in a subject; a proximal portion from which the elongate member extends distally, the proximal portion sized to be maintained outside of the subject, the proximal portion including a rotatable component in rotational operation with an impeller in a pump portion of the elongate member, the rotatable component including a portion of a guidewire pathway, and wherein rotation of the component causes mis-alignment or alignment between the portion of the guidewire pathway and a second portion of the guidewire pathway formed in a second component of the proximal portion that is not in rotational operation with the impeller. This aspect may further include any other suitable feature or element described herein.

One aspect of the disclosure is an intravascular blood pump, comprising: an elongate member including a blood pump, the elongate member sized for intravascular positioning in a subject; a proximal portion from which the elongate member extends distally, the proximal portion sized to be maintained outside of the subject, the proximal portion including a guidewire access port, the guidewire access port configured for coupling to a connector of a fluid line when the guidewire is not in the access port such that fluid can be delivered into the guidewire port from the fluid line or received from the guidewire port into the fluid line.

In this aspect, the guidewire port may be disposed in a radially side surface of the proximal portion. The connector may comprises a luer fitting.

In this aspect, the guidewire port may be part of a guidewire pathway that extends distally beyond a distal end of an impeller in a pump portion of the blood pump.

In this aspect, a guidewire path may include the guidewire port, the guidewire path further including a portion with a curved configuration, wherein the curved configuration is the proximal portion.

This aspect may further comprise any other suitable feature or element described herein.

One aspect of this disclosure is an intravascular blood pump, comprising: an expandable blood flow conduit with a distal end and a proximal end, and at least one impeller disposed radially within the conduit, the conduit having a central region, a proximal region proximal to the central region, and a distal region distal to the central region, wherein the central region has greater flexibility than both the proximal region and the distal region, the distal and proximal regions between the conduit proximal end and distal end.

In this aspect, the impeller can be a proximal impeller and is radially within the proximal region, the blood pump further comprising a distal impeller distal to the proximal impeller, the distal impeller radially within the distal region, and wherein the distal impeller and the proximal impeller do not extend axially into the central region.

In this aspect, the conduit can includes a support structure, optionally extending an entire length of the fluid lumen.

This aspect can further comprise any other suitable feature or element described herein.

This aspect includes a method of positioning a pump in this aspect, the method including positioning the distal region distal to an aortic valve and the proximal region proximal to the valve.

One aspect of the disclosure is an intravascular blood pump, the pump including an outer conduit having a distal end and a proximal end, the pump including a support structure comprising a plurality of elongate elements disposed in a proximal region of the support structure, the plurality of elongate elements each having a transition portion where the respective arm transitions from a larger diameter region to a smaller diameter region, wherein in the transition portion each of the respective arms has a vertical section, relative to a longitudinal axis of the outer housing; and an impeller at least partially disposed within the conduit.

In this aspect, the plurality of elongate elements have a bend region adjacent the vertical section, wherein the bend transitions the vertical section to one of the larger diameter region and the smaller diameter region. The plurality of elongate elements may have a second bend region adjacent the vertical section, where the second bend region transitions the vertical section to the other of the larger diameter region and the smaller diameter region.

In this aspect, the plurality of elongate elements are configured such that they do not influence or modify the fluid outflow in any meaningful way, as would be understood by one or ordinary skill in the art. This aspect may further comprise any other suitable feature or element described herein.

One aspect of the disclosure is an intravascular blood pump, comprising: an outer blood flow conduit defining a fluid lumen having a distal end and a proximal end, the pump also including a support structure comprising a proximal region comprising a plurality of peaks pointing in a proximal direction, wherein a first set of the plurality of peaks extends to a first axial location and a second set of the plurality of peaks extends further proximally than the first axial location; and an impeller radially disposed within the fluid lumen.

In this aspect, the second set of the plurality of peaks may be integrally formed with a plurality of proximal struts that each have a vertical section which transitions the struts from a larger diameter section to a smaller diameter section. This aspect may include any other suitable feature or element described herein.

One aspect of the disclosure is an intravascular blood pump, comprising: an outer expandable blood flow conduit defining a fluid lumen having a distal end and a proximal end, at least one of the distal end and the proximal end of the fluid lumen having a radially outward flared configuration, the pump further including a support structure, the pump portion also including an impeller radially disposed within the outer expandable blood flow conduit.

In this aspect, the support structure may include a plurality of struts, and wherein the proximal end of the fluid lumen is flared and is disposed distally to a transition region in each of the plurality of struts.

In this aspect, the proximal end of the fluid lumen can be flared, and a proximal end of the impeller extends further proximally than the proximal end of the lumen.

In this aspect, the at least one flared configuration may be supported by the support structure.

This aspect may further include any other suitable feature or element described herein.

One aspect of this disclosure is an intravascular blood pump, comprising: an outer expandable conduit defining a fluid lumen having a distal end and a proximal end, the pump also including a support structure with a first portion having a proximal end and a distal end, the first portion including a plurality of elongate elements, each of the plurality of elongate elements having a helical configuration; and an impeller radially disposed within the fluid lumen.

In this aspect, the first portion may axially overlap with at least a portion of the impeller.

In this aspect, the first portion may completely axially overlap with the impeller.

In this aspect, in a side view, each of the plurality of elongate elements may follow a helical configuration of at least one impeller blade, wherein follows in this regard does not require that the helical configuration have the same pitch as the at least one impeller blade.

In this aspect, in a side view, the plurality of elongate elements may form a greater angle with a longitudinal axis of the fluid lumen that do the plurality of elongate elements in sections just proximal to and just distal to the first portion (i.e., greater curvature in the first portion, relative to the longitudinal axis).

In this aspect, in a side view, each of the elongate elements may have a tangent that forms an angle of 45 degrees or less with a tangent of a helical impeller blade, optionally 35 degrees or less, optionally 20 degrees or less, optionally 15 degrees or less, optionally 10 degrees or less (see FIG. 23).

In this aspect, in a side view, each of the elongate elements may have a tangent that forms an angle of 45 degrees or less with a camber line of the helical blade where the elongate element and helical blade axially overlap, optionally 35 degrees or less, optionally 20 degrees or less, optionally 15 degrees or less, optionally 10 degrees or less (e.g., see FIG. 23).

In this aspect, at least one of the plurality of elongate elements that has the helical configuration does not make a complete turn (i.e., 360 degrees in an end view) around the support structure.

In this aspect, at least one of the plurality of elongate elements with the helical configuration may make a complete turn (i.e., 360 degrees in an end view) around the support structure.

In this aspect, the support structure may further comprise a second portion with a second plurality of elongate elements, each of the second plurality of elongate elements may have a helical configuration. The second portion may be axially spaced from the first portion. The second portion may at least partially axially overlap with the impeller. The second portion may at least partially overlaps with a second impeller that is axially spaced from the impeller. The second portion may have the same configuration as the first portion.

In this aspect, the elongate elements may each be connecting elements to adjacent sections of the support structure.

In this aspect, the elongate elements may each have a first end coupled to a first adjacent section of the support structure and a second end coupled to a second adjacent section of the support structure.

In this aspect, the first portion may have an axial length between 1 and 20 mm.

In this aspect, the first portion may have an axial length that is between 1 and 100 % of the length of the impeller, optionally between 1 and 80%, optionally between 1 and 70%, optionally between 1 and 60%, optionally between 1 and 50%, optionally between 1 and 40%, optionally between 1 and 30%.

In this aspect, the first portion may overlap axially with the impeller along at least 100% of the impeller length, optionally no more than 90% of its length, optionally no more than 80%, optionally no more than 70%, optionally no more than 60%, optionally no more than 50%, optionally no more than 40%, optionally no more than 30%.

One aspect of the disclosure is a method of collapsing a pump portion of a blood pump, comprising: rotating an elongate member to which a collapsible pump portion is secured when the elongate member is disposed within a patient.

In this aspect, the rotating may facilitate the collapse of one or more blades of an impeller in the pump portion, optionally helical blades.

In this aspect, the rotating may cause a support structure of the pump portion to apply forces on one or more blades (optionally helical blades) of an impeller in the pump portion.

In this aspect, the method may further comprise also applying a tensile force on the elongate member, either simultaneously with the rotating or occurring at distinct times.

One aspect of the disclosure is a method of collapsing an impeller of a pump portion of a blood pump, comprising: collapsing a support structure in which an impeller is disposed, wherein the collapsing step applies a radially inward collapsing force on a helical blade of the impeller with a elongate member of the support structure that has a helical configuration (optionally integrally formed with the rest of the scaffold pattern, i.e., not a separate component coupled to the scaffold pattern), wherein the radially inward collapsing force from the elongate member with the helical configuration ensures that the helical blade collapses in a particular direction relative to a central support structure, optionally a hub.

In this aspect, the collapsing step may apply a radially inward collapsing forces on the helical blade of the impeller with a plurality of elongate members of the support structure that each have helical configuration.

In this aspect, the collapsing step may comprise applying at least one of a tensioning force and a rotational force to an elongate member to which the support structure is coupled.

In this aspect, the method may further comprise any other suitable method step herein.

One aspect of this disclosure is an intravascular blood pump, comprising: an outer expandable blood flow conduit defining a fluid lumen having a distal end and a proximal end; an impeller disposed radially within the fluid lumen, the impeller coupled to a rotatable shaft; a rotatable drive member (e.g., a drive cable) in operational communication with the rotatable shaft, the rotatable drive member rotatable in response to an energy source (e.g., a motor); and a speed increaser operably interacting with the drive member and the rotatable shaft, the speed increaser causing the rotatable shaft to rotate faster than the rotatable drive member.

In this aspect, the speed increaser may comprise a first gear coupled to the drive member and a second gear coupled to the rotatable shaft, the first and second gears interfacing each other, the second gear having a smaller diameter than the first gear.

In this aspect, the rotatable drive member may be co-axial with the rotatable shaft.

In this aspect, the rotatable drive member may not be co-axial with the rotatable shaft.

In this aspect, the speed increaser may comprise a planetary gear box.

In this aspect, the speed increaser may comprise a shaft different than the drive member and the rotatable shaft.

### BRIEF DESCRIPTION OF THE DRAWINGS

FIG. 1 is a side view of an exemplary pump portion that includes a conduit, a plurality of impellers, an expandable member

FIG. 2 is a side view of an exemplary pump portion that includes a conduit, a plurality of impellers, and a plurality of expandable members.

FIGS. 3A, 3B, 3C and 3D illustrate an exemplary pump portion that includes a conduit, a plurality of impellers, and a plurality of expandable members.

FIG. 4 illustrates an exemplary placement of a pump portion, the pump portion including a conduit, a plurality of expandable members, and a plurality of impellers.

FIG. 5 illustrates an exemplary pump portion.

FIG. 6A illustrates at least a portion of an exemplary medical device that has a pump portion, where at least two different impellers can be rotated at different speeds.

FIG. 6B illustrates at least a portion of an exemplary medical device that has a pump portion, where at least two different impellers can be rotated at different speeds.

FIG. 6C illustrates at least a portion of an exemplary medical device that has a pump portion with at least two impellers with different pitches.

FIG. 7 illustrates at least a portion of an exemplary medical device that has a pump portion.

FIG. 8 illustrates a pump portion with multiple impellers, with a bend formed therein between adjacent impellers.

FIG. 9 illustrates a pump portion with a plurality of impellers.

FIG. 10 shows a side view of a portion of a fluid movement device, including a pump portion thereof, that includes a central, or intermediate, member axially spaced between first and second impellers.

FIGS. 11A, 11B and 11C illustrates an exemplary central, or intermediate, member.

FIGS. 12A, 12B, 12C and 12D illustrate different views of an exemplary medical device, including a pump portion or a portion of a pump portion, that includes a first central (intermediate) member and a second central (intermediate) member.

FIGS. 13A, 13B and 13C illustrate different view of an exemplary central, or intermediate, member.

FIGS. 14A and 14B illustrate an exemplary blood pump in which at least the components that are shown are non-collapsible and are not collapsed for delivery.

FIGS. 15A-D illustrates an exemplary blood pump that includes a guidewire pathway and at least one fluid purge pathway.

FIGS. 16A and B illustrates an exemplary blood pump that includes a guidewire pathway and at least two fluid purge pathways that are not in fluid communication.

FIGS. 17A-F illustrate an exemplary pump portion that includes an expandable housing, including an exemplary scaffold design.

FIGS. 18A and B illustrate an exemplary scaffold design.

FIGS. 19A and 19B illustrate an exemplary scaffold design.

FIG. 20 illustrates an exemplary scaffold design.

FIGS. 21A-C illustrate an exemplary scaffold design.

FIGS. 22A-B illustrate an exemplary scaffold design.

FIG. 23 illustrates a portion of an expandable housing with a fluid lumen that has an outwardly flared configuration at at least one end of the fluid lumen.

FIG. 24 illustrates an outer housing that includes one or more blades extending radially inward.

FIG. 25 illustrates a proximal portion of an exemplary pump portion, wherein the pump portion includes one or blades that are sized and configured to interface with a portion of the scaffold.

FIGS. 26A and 26B illustrate portions of exemplary blood pumps with exemplary speed increaser assemblies and mechanisms that may be incorporated into a blood pump.

FIG. 27 illustrates an exemplary pump portion that is adapted, optionally with at least one tensioning member, so that the pump portion can be deflected at at least one location along the length of the pump portion.

FIGS. 28A-28F illustrate an exemplary sequence of steps that may be carried out based on an exemplary method of using an exemplary blood pump.

FIGS. 29A and 29B illustrate exemplary collapsible flow modifying elements that may be part of a pump portion, optionally disposed between two collapsible impellers.

FIG. 30A illustrates an exemplary blood flow conduit support member that includes a plurality of apertures therein, the apertures sized and configured to receive, and interface with, one or more flow modifying elements.

FIG. 30B illustrates an end view of an exemplary support member, wherein a plurality of flow modifying elements are each disposed in one of a plurality of support member apertures.

FIGS. 31A and 31B illustrate exemplary flow modifying elements, which may be sized and configured to be advanced through a conduit support member aperture, such as those in FIGS. 30A and 30B.

FIGS. 32A, 32B and 32C illustrate an exemplary conduit support member that includes a plurality of flow modifying elements that are integrally formed with the support member, and that can self-deploy to an active, flow modifying configuration in which are extend radially inward.

FIGS. 33A and 33B illustrate an exemplary pump portion that includes a plurality of flow modifying elements 445, optionally secured to struts of the pump portion.

FIG. 34 illustrates a side view of exemplary flow modifying elements disposed between first and second impellers.

FIGS. 35A and 35B illustrate side and top views, respectively, of exemplary flow modifying elements (e.g., diffusers in these figures). FIG. 35B illustrates the configuration of the diffusers in the top view, as well as the fluid direction relative to the diffuser.

### DETAILED DESCRIPTION

The present disclosure is related to medical devices, systems, and methods of use and manufacture. Medical devices herein may include a pump portion adapted and configured to be disposed within a physiologic vessel, wherein the pump includes one or more components that act upon fluid. For example, pump portions herein may include one or more impellers that are configured such that when rotated, they facilitate the movement of a fluid such as blood.

FIG. 1 is a side view illustrating a distal portion of an exemplary intravascular fluid pump, including pump portion 1600, wherein pump portion 1600 includes proximal impeller 1606 and distal impeller 1616, both of which are in operable communication with drive cable 1612. Pump portion 1600 is in an expanded configuration in FIG. 1, but is adapted to be collapsed to a delivery configuration so that it can be delivered with a lower profile. The impellers can in rotational communication with drive cable 1612, directly or indirectly. Drive cable 1612 is in operable communication with an external motor, not shown, and extends through elongate shaft 1610. The phrases “pump portion” and “working portion” (or derivatives thereof) may be used herein interchangeably unless indicated to the contrary. For example without limitation, “pump portion” 1600 can also be referred to herein as a “working portion.”

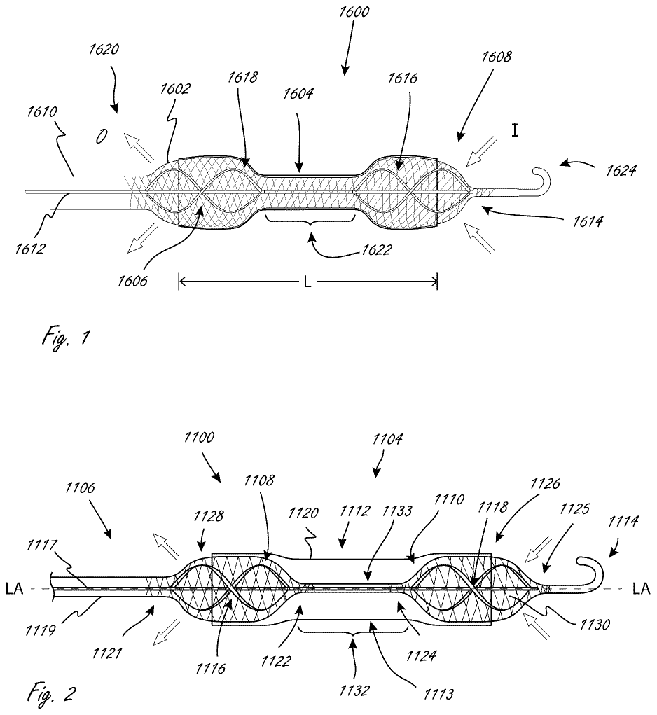

FIG. 2 is a side view illustrating a deployed configuration (shown extracorporally) of a distal portion of an exemplary embodiment of a fluid movement system. Exemplary system 1100 includes pump portion 1104 (which as set forth herein may also be referred to herein as a pump portion) and an elongate portion 1106 extending from pump portion 1104. Elongate portion 1106 can extend to a more proximal region of the system, not shown for clarity, and that can include, for example, a motor. Pump portion 1104 includes first expandable member 1108 and second expandable member 1110, axially spaced apart along a longitudinal axis LA of pump portion 1104. Spaced axially in this context refers to the entire first expandable member being axially spaced from the entire second expandable member along a longitudinal axis LA of pump portion 1104. A first end 1122 of first expandable member 1108 is axially spaced from a first end 1124 of second expandable member 1110. Some “expandable members” herein may also be referred to herein as baskets.

First and second expandable members 1108 and 1110 generally each include a plurality of elongate segments disposed relative to one another to define a plurality of apertures 1130, only one of which is labeled in the second expandable member 1110. The expandable members can have a wide variety of configurations and can be constructed in a wide variety of ways, such as any of the configurations or constructions in, for example without limitation, U.S. Pat. No. 7,841,976, or the tube in U.S. Pat. No. 6,533,716, which is described as a self-expanding metal endoprosthetic material. For example, without limitation, one or both of the expandable members can have a braided construction or can be at least partially formed by laser cutting a tubular element.

Pump portion 1104 also includes blood flow conduit 1112, which in this embodiment is supported by first expandable member 1108 and to second expandable member 1110. Conduit 1112 also extends axially in between first expandable member 1108 and second expandable member 1110 in the deployed configuration. A central region 1113 of conduit 1112 spans an axial distance 1132 where the pump portion is void of first and second expandable members 1108 and 1110. Central region 1113 can be considered to be axially in between the expandable members. Distal end 1126 of conduit 1112 does not extend as far distally as a distal end 1125 of second expandable member 1110, and proximal end of conduit 1128 does not extend as far proximally as proximal end 1121 of first expandable member 1108.

When the disclosure herein refers to a conduit being coupled to an expandable member, the term coupled in this context does not require that the conduit be directly attached to the expandable member so that conduit physically contacts the expandable member. Even if not directly attached, however, the term coupled in this context refers to the conduit and the expandable member being joined together such that as the expandable member expands or collapses, the conduit also begins to transition to a different configuration and/or size. Coupled in this context therefore refers to conduits that will move when the expandable member to which it is coupled transitions between expanded and collapsed configurations. The conduits herein are considered to create a pathway for fluid to be moved, and may be defined by a one or more components of the pump portion.

Any of the conduits herein can be deformable to some extent. For example, conduit 1112 includes elongate member 1120 that can be made of one or more materials that allow the central region 1113 of conduit to deform to some extent radially inward (towards LA) in response to, for example and when in use, forces from valve tissue (e.g., leaflets) or a replacement valve as pump portion 1104 is deployed towards the configuration shown in FIG. 2. The conduit may be stretched tightly between the expandable members in some embodiments. The conduit may alternatively be designed with a looseness that causes a greater degree of compliance. This can be desirable when the pump portion is disposed across fragile structures such as an aortic valve, which may allow the valve to compress the conduit in a way that minimizes point stresses in the valve. In some embodiments, the conduit may include a membrane attached to the proximal and distal expandable members. Exemplary materials that can be used for any conduits herein include, without limitations, polyurethane rubber, silicone rubber, acrylic rubber, expanded polytetrafluoroethylene, polyethylene, polyethylene terephthalate, including any combination thereof.

Any of the conduits herein can have a thickness of, for example, 0.5-20 thousandths of an inch (thou), such as 1-15 thou, or 1.5 to 15 thou, 1.5 to 10 thou, or 2 to 10 thou.

Any of the conduits herein, or at least a portion of the conduit, can be impermeable to blood. In FIG. 2, pump portion 1104 includes a lumen that extends from distal end 1126 of conduit 1112 and extends to proximal end 1128 of conduit 1112. The lumen is defined by conduit 1112 in central region 1113, but can be thought of being defined by both the conduit and portions of the expandable members in regions axially adjacent to central region 1113. In this embodiment, however, it is the conduit material that causes the lumen to exist and prevents blood from passing through the conduit.

Any of the conduits herein that are secured to one or more expandable members can be, unless indicated to the contrary, secured so that the conduit is disposed radially outside of one or more expandable members, radially inside of one or more expandable members, or both, and the expandable member can be impregnated with the conduit material.

The proximal and distal expandable members help maintain the conduit in an open configuration by providing radial support for the conduit, while each also creates a working environment for an impeller, described below. Each of the expandable members, when in the deployed configuration, is maintained in a spaced relationship relative to a respective impeller, which allows the impeller to rotate within the expandable member without contacting the expandable member. Pump portion 1104 includes first impeller 1116 and second impeller 1118, with first impeller 1116 disposed radially within first expandable member 1108 and second impeller 1118 disposed radially within second expandable member 1110. In this embodiment, the two impellers even though they are distinct and separate impellers, are in operable communication with a common drive mechanism (e.g., drive cable 1117), such that when the drive mechanism is activated the two impellers rotate together. In this deployed configuration, impellers 1116 and 1118 are axially spaced apart along longitudinal axis LA, just as are the expandable members 1108 and 1110 are axially spaced apart.

Impellers 1116 and 1118 are also axially within the ends of expandable members 1108 and 1110, respectively (in addition to being radially within expandable members 1108 and 1110). The impellers herein can be considered to be axially within an expandable member even if the expandable member includes struts extending from a central region of the expandable member towards a longitudinal axis of the pump portion (e.g., tapering struts in a side view). In FIG. 2, second expandable member 1110 extends from first end 1124 (proximal end) to second end 1125 (distal end).

In FIG. 2, a distal portion of impeller 1118 extends distally beyond distal end 1126 of conduit 1112, and a proximal portion of impeller 1116 extends proximally beyond proximal end 1128 of conduit 1112. In this figure, portions of each impeller are axially within the conduit in this deployed configuration.

In the exemplary embodiment shown in FIG. 2, impellers 1116 and 1118 are in operable communication with a common drive mechanism 1117, and in this embodiment, the impellers are each coupled to drive mechanism 1117, which extends through shaft 1119 and pump portion 1104. Drive mechanism 1117 can be, for example, an elongate drive cable, which when rotated causes the impellers to rotate. In this example, as shown, drive mechanism 1117 extends to and is axially fixed relative to distal tip 1114, although it is adapted to rotate relative to distal tip 1114 when actuated. Thus, in this embodiment, the impellers and drive mechanism 1117 rotate together when the drive mechanism is rotated. Any number of known mechanisms can be used to rotate drive mechanism, such as with a motor (e.g., an external motor).

The expandable members and the conduit are not in rotational operable communication with the impellers and the drive mechanism. In this embodiment, proximal end 1121 of proximal expandable member 1108 is coupled to shaft 1119, which may be a shaft of elongate portion 1106 (e.g., an outer catheter shaft). Distal end 1122 of proximal expandable member 1108 is coupled to central tubular member 1133, through which drive mechanism 1117 extends. Central tubular member 1133 extends distally from proximal expandable member 1108 within conduit 1112 and is also coupled to proximal end 1124 of distal expandable member 1110. Drive mechanism 1117 thus rotates within and relative to central tubular member 1133. Central tubular member 1133 extends axially from proximal expandable member 1108 to distal expandable member 1110. Distal end 1125 of distal expandable member 1110 is coupled to distal tip 1114, as shown. Drive mechanism 1117 is adapted to rotate relative to tip 1114, but is axially fixed relative to tip 1114.

Pump portion 1104 is adapted and configured to be collapsed to a smaller profile than its deployed configuration (which is shown in FIG. 2). This allows it to be delivered using a lower profile delivery device (smaller French size) than would be required if none of pump portion 1104 was collapsible. Even if not specifically stated herein, any of the expandable members and impellers may be adapted and configured to be collapsible to some extent to a smaller delivery configuration.

The pump portions herein can be collapsed to a collapsed delivery configuration using conventional techniques, such as with an outer sheath that is movable relative to the pump portion (e.g., by axially moving one or both of the sheath and pump portion). For example without limitation, any of the systems, devices, or methods shown in the following references may be used to facilitate the collapse of a pump portion herein: U.S. Pat. No. 7,841,976 or U.S. Pat. No. 8,052,749, the disclosures of which are incorporated by reference herein for all purposes.

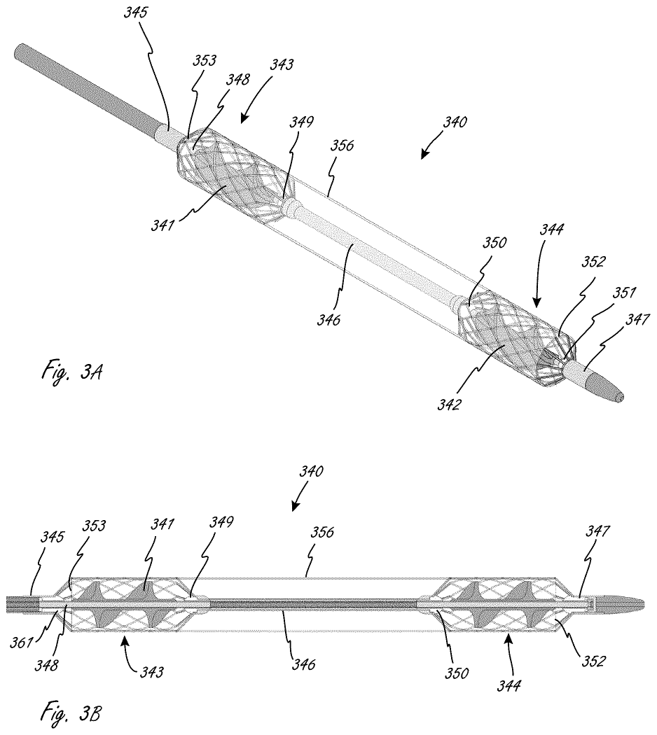

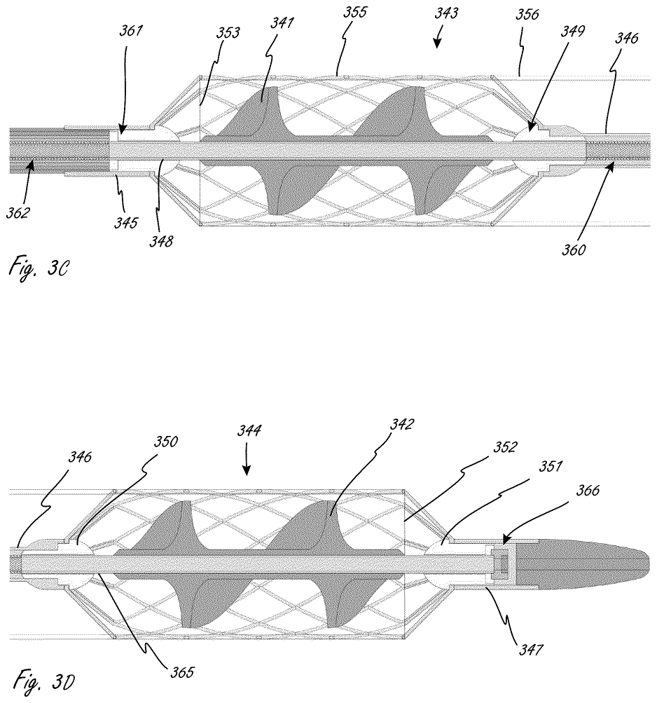

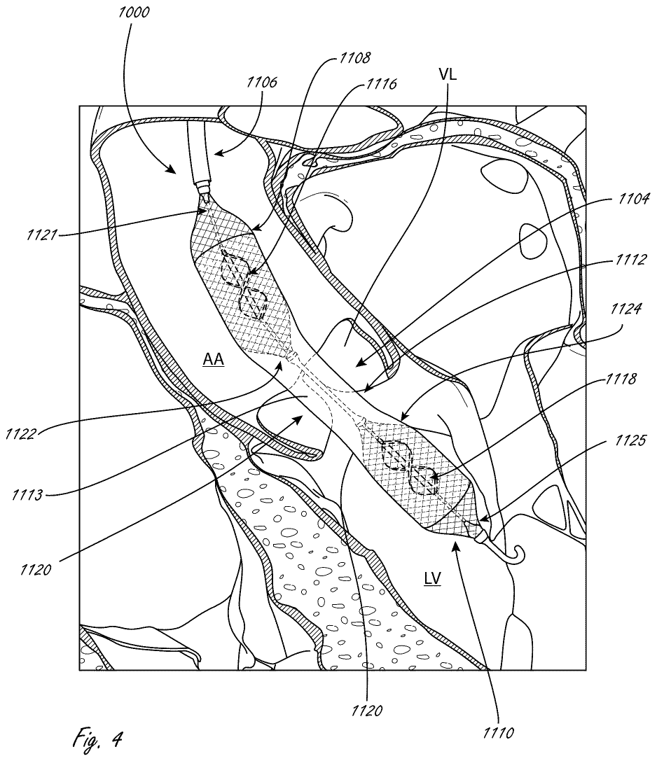

FIGS. 3A-3E show an exemplary pump portion that is similar in some ways to the pump portion shown in FIG. 2. Pump portion 340 is similar to pump portion 1104 in that in includes two expandable members axially spaced from one another when the pump portion is expanded, and a conduit extending between the two expandable members. FIG. 3A is a perspective view, FIG. 3B is a side sectional view, and FIGS. 3C and 3D are close-up side sectional views of sections of the view in FIG. 3B.

Pump portion 340 includes proximal impeller 341 and distal impeller 342, which are coupled to and in operational communication with a drive cable, which defines therein a lumen. The lumen can be sized to accommodate a guidewire, which can be used for delivery of the pump portion to the desired location. The drive cable, in this embodiment, includes first section 362 (e.g., wound material), second section 348 (e.g., tubular member) to which proximal impeller 341 is coupled, third section 360 (e.g., wound material), and fourth section 365 (e.g., tubular material) to which distal impeller 342 is coupled. The drive cable sections all have the same inner diameter, so that lumen has a constant inner diameter. The drive cable sections can be secured to each other using known attachment techniques. A distal end of fourth section 365 extends to a distal region of the pump portion, allowing the pump portion to be, for example, advanced over a guidewire for positioning the pump portion. In this embodiment the second and fourth sections can be stiffer than first and third sections. For example, second and fourth can be tubular and first and third sections can be wound material to impart less stiffness.

Pump portion 340 includes a blood flow conduit, proximal expandable member 343 and distal expandable member 344, each of which extends radially outside of one of the impellers. The expandable members have distal and proximal ends that also extend axially beyond distal and proximal ends of the impellers, which can be seen in FIGS. 3B-3D. That pump also includes conduit 356, which has a proximal end 353 and a distal end 352. The two expandable members each include a plurality of proximal struts and a plurality of distal struts. The proximal struts in proximal expandable member 343 extend to and are secured to shaft section 345, which is coupled to bearing 361, through which the drive cable extends and is configured and sized to rotate. The distal struts of proximal expandable member 343 extend to and are secured to a proximal region (to a proximal end in this case) of central tubular member 346, which is disposed axially in between the expandable members. The proximal end of central tubular member 346 is coupled to bearing 349, as shown in FIG. 3C, through which the drive cable extends and rotates. The proximal struts of distal expandable member 344 extend to and secured to a distal region (to a distal end in this case) of central tubular member 346. Bearing 350 is also coupled to the distal region of central tubular member 346, as is shown in FIG. 3D. The drive cable extends through and rotates relative to bearing 350. Distal struts of distal expandable member extend to and are secured to shaft section 347 (see FIG. 3A), which can be considered part of the distal tip. Shaft section 347 is coupled to bearing 351 (see FIG. 3D), through which the drive cable extends and rotates relative to. The distal tip also includes bearing 366 (see FIG. 3D), which can be a thrust bearing. Working portion 340 can be similar to or the same in some aspects to working portion 1104, even if not explicitly included in the description. In this embodiment, conduit 356 extends at least as far as ends of the impeller, unlike in working portion 1104. Either embodiment can be modified so that the conduit extends to a position as set forth in the other embodiment. In some embodiments, section 360 can be a tubular section instead of wound.

In alternative embodiments, at least a portion of any of the impellers herein may extend outside of the fluid lumen. For example, only a portion of an impeller may extend beyond an end of the fluid lumen in either the proximal or distal direction. In some embodiments, a portion of an impeller that extends outside of the fluid lumen is a proximal portion of the impeller, and includes a proximal end (e.g., see the proximal impeller in FIG. 2). In some embodiments, the portion of the impeller that extends outside of the fluid lumen is a distal portion of the impeller, and includes a distal end (e.g., see the distal impeller in FIG. 2). When the disclosure herein refers to impellers that extend outside of the fluid lumen (or beyond an end), it is meant to refer to relative axial positions of the components, which can be most easily seen in side views or top views, such as in FIG. 2.

A second impeller at another end of the fluid lumen may not, however, extend beyond the fluid lumen. For example, an illustrative alternative design can include a proximal impeller that extends proximally beyond a proximal end of the fluid lumen (like the proximal impeller in FIG. 2), and the fluid lumen does not extend distally beyond a distal end of a distal impeller (like in FIG. 3B). Alternatively, a distal end of a distal impeller can extend distally beyond a distal end of the fluid lumen, but a proximal end of a proximal impeller does not extend proximally beyond a proximal end of the fluid lumen. In any of the pump portions herein, none of the impellers may extend beyond ends of the fluid lumen.

While specific exemplary locations may be shown herein, the fluid pumps may be able to be used in a variety of locations within a body. Some exemplary locations for placement include placement in the vicinity of an aortic valve or pulmonary valve, such as spanning the valve and positioned on one or both sides of the valve, and in the case of an aortic valve, optionally including a portion positioned in the ascending aorta. In some other embodiments, for example, the pumps may be in use, positioned further downstream, such as being disposed in a descending aorta.

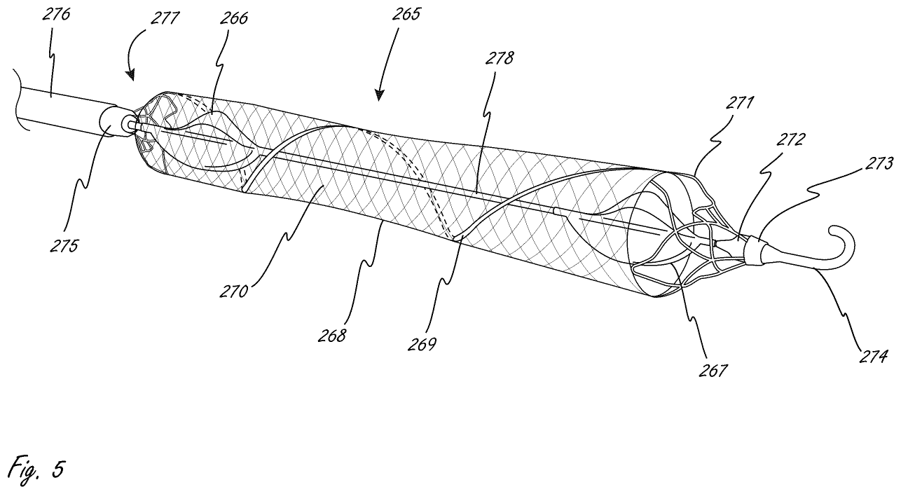

FIG. 4 illustrates an exemplary placement of pump portion 1104 from system 1000 from FIG. 2, and also illustrates an exemplary placement location for any of the pump portions herein. One difference shown in FIG. 4 is that the conduit extends at least as far as the ends of the impellers, like in FIGS. 3A-3D. FIG. 4 shows pump portion 1104 in a deployed configuration, positioned in place across an aortic valve. Pump portion 1104 can be delivered as shown via, for example without limitation, femoral artery access (a known access procedure). While not shown for clarity, system 1000 can also include an outer sheath or shaft in which pump portion 1104 is disposed during delivery to a location near an aortic valve. The sheath or shaft can be moved proximally (towards the ascending aorta “AA” and away from left ventricle “LV”) to allow for deployment and expansion of pump portion 1104. For example, the sheath can be withdrawn to allow for expansion of second expandable member 1110, with continued proximal movement allowing first expandable member 1108 to expand.

In this embodiment, second expandable member 1110 has been expanded and positioned in a deployed configuration such that distal end 1125 is in the left ventricle “LV,” and distal to aortic valve leaflets “VL,” as well as distal to the annulus. Proximal end 1124 has also been positioned distal to leaflets VL, but in some methods proximal end 1124 may extend slightly axially within the leaflets VL. This embodiment is an example of a method in which at least half of the second expandable member 1110 is within the left ventricle, as measured along its length (measured along the longitudinal axis). And as shown, this is also an example of a method in which the entire second expandable member 1110 is within the left ventricle. This is also an example of a method in which at least half of second impeller 1118 is positioned within the left ventricle, and also an embodiment in which the entire second impeller 1118 is positioned within the left ventricle.

Continued retraction of an outer shaft or sheath (and/or distal movement of working end 1104 relative to an outer sheath or shaft) continues to release conduit 1112, until central region 1113 is released and deployed. The expansion of expandable members 1108 and 1110 causes conduit 1112 to assume a more open configuration, as shown in FIG. 4. Thus, while in this embodiment conduit 1112 does not have the same self-expanding properties as the expandable members, the conduit will assume a deployed, more open configuration when the working end is deployed. At least a portion of central region 1113 of conduit 1112 is positioned at an aortic valve coaptation region. In FIG. 3, there is a short length of central region 1113 that extends distally beyond the leaflets VL, but at least some portion of central region 1113 is axially within the leaflets.

Continued retraction of an outer shaft or sheath (and/or distal movement of working end 1104 relative to an outer sheath or shaft) deploys first expandable member 1108. In this embodiment, first expandable member 1108 has been expanded and positioned (as shown) in a deployed configuration such that proximal end 1121 is in the ascending aorta AA, and proximal to leaflets “VL.” Distal end 1122 has also been positioned proximal to leaflets VL, but in some methods distal end 1122 may extend slightly axially within the leaflets VL. This embodiment is an example of a method in which at least half of first expandable member 1110 is within the ascending aorta, as measured along its length (measured along the longitudinal axis). And as shown, this is also an example of a method in which the entire first expandable member 1110 is within the AA. This is also an example of a method in which at least half of first impeller 1116 is positioned within the AA, and also an embodiment in which the entire first impeller 1116 is positioned within the AA.

At any time during or after deployment of pump portion 1104, the position of the pump portion can be assessed in any way, such as under fluoroscopy. The position of the pump portion can be adjusted at any time during or after deployment. For example, after second expandable member 1110 is released but before first expandable member 1108 is released, pump portion 1104 can be moved axially (distally or proximally) to reposition the pump portion. Additionally, for example, the pump portion can be repositioned after the entire working portion has been released from a sheath to a desired final position.

It is understood that the positions of the components (relative to the anatomy) shown in FIG. 4 are considered exemplary final positions for the different components of working portion 1104, even if there was repositioning that occurred after initial deployment.

The one or more expandable members herein can be configured to be, and can be expanded in a variety of ways, such as via self-expansion, mechanical actuation (e.g., one or more axially directed forces on the expandable member, expanded with a separate balloon positioned radially within the expandable member and inflated to push radially outward on the expandable member), or a combination thereof.

Expansion as used herein refers generally to reconfiguration to a larger profile with a larger radially outermost dimension (relative to the longitudinal axis), regardless of the specific manner in which the one or more components are expanded. For example, a stent that self-expands and/or is subject to a radially outward force can “expand” as that term is used herein. A device that unfurls or unrolls can also assume a larger profile, and can be considered to expand as that term is used herein.

The impellers can similarly be adapted and configured to be, and can be expanded in a variety of ways depending on their construction. For examples, one or more impellers can, upon release from a sheath, automatically revert to or towards a different larger profile configuration due to the material(s) and/or construction of the impeller design (see, for example, U.S. Pat. No. 6,533,716, or U.S. Pat. No. 7,393,181, both of which are incorporated by reference herein for all purposes). Retraction of an outer restraint can thus, in some embodiments, allow both the expandable member and the impeller to revert naturally to a larger profile, deployed configuration without any further actuation.

As shown in the example in FIG. 4, the pump portion includes first and second impellers that are spaced on either side of an aortic valve, each disposed within a separate expandable member. This is in contrast to some designs in which a working portion includes a single elongate expandable member. Rather than a single generally tubular expandable member extending all the way across the valve, working end 1104 includes a conduit 1112 extending between expandable members 1108 and 1110. The conduit is more flexible and deformable than the pump at the locations of the impellers, which can allow for more deformation of the pump portion at the location of the leaflets than would occur if an expandable member spanned the aortic valve leaflets. Having a more flexible central region may also cause less damage to the leaflets after the pump portion has been deployed in the subject.

Additionally, forces on a central region of a single expandable member from the leaflets might translate axially to other regions of the expandable member, perhaps causing undesired deformation of the expandable member at the locations of the one or more impellers. This may cause the outer expandable member to contact the impeller, undesirably interfering with the rotation of the impeller. Designs that include separate expandable members around each impeller, particularly where each expandable member and each impeller are supported at both ends (i.e., distal and proximal), result in a high level of precision in locating the impeller relative to the expandable member. Two separate expandable members may be able to more reliably retain their deployed configurations compared with a single expandable member.

As described herein above, it may be desirable to be able to reconfigure the working portion so that it can be delivered within a 9F sheath and still obtain high enough flow rates when in use, which is not possible with some products currently in development and/or testing. For example, some products are too large to be able to reconfigured to a small enough delivery profile, while some smaller designs may not be able to achieve the desired high flow rates. An exemplary advantage of the examples in FIGS. 1, 2, 3A-3D and 4 is that, for example, the first and second impellers can work together to achieve the desired flow rates, and by having two axially spaced impellers, the overall working portion can be reconfigured to a smaller delivery profile than designs in which a single impeller is used to achieved the desired flow rates. These embodiments thus use a plurality of smaller, reconfigurable impellers that are axially spaced to achieve both the desired smaller delivery profile as well as to achieve the desired high flow rates.

Embodiments herein can thus achieve a smaller delivery profile while maintaining sufficiently high flow rates, while creating a more deformable and flexible central region of the working portion, the exemplary benefits of which are described above (e.g., interfacing with delicate valve leaflets).

FIG. 5 illustrates a working portion that is similar to the working portion shown in FIG. 1. Working portion 265 includes proximal impeller 266, distal impeller 267, both of which are coupled to drive shaft 278, which extends into distal bearing housing 272. There is a similar proximal bearing housing at the proximal end of the working portion. Working portion also includes expandable member, referred to 270 generally, and conduit 268 that is secured to the expandable member and extends almost the entire length of expandable member. Expandable member 270 includes distal struts 271 that extend to and are secured to strut support 273, which is secured to distal tip 273. Expandable member 270 also includes proximal struts there are secured to a proximal strut support. All features similar to that shown in FIG. 1 are incorporated by reference for all purposes into this embodiment even if not explicitly stated. Expandable member 265 also includes helical tension member 269 that is disposed along the periphery of the expandable member, and has a helical configuration when the expandable member is in the expanded configuration as shown. The helical tension member 269 is disposed and adapted to induce rotation wrap upon collapse. Working portion 265 can be collapsed from the shown expanded configuration while simultaneously rotating one or both impellers at a relatively slow speed to facilitate curled collapse of the impellers due to interaction with the expandable member.

There are alternative ways to construct the pump portion to cause rotation of the expandable member upon collapse by elongation (and thus cause wrapping and collapse of the impeller blades). Any expandable member can be constructed with this feature, even in dual-impeller designs. For example, with an expandable member that includes a plurality of “cells,” as that term is commonly known (e.g., a laser cut elongate member), the expandable member may have a plurality of particular cells that together define a particular configuration such as a helical configuration, wherein the cells that define the configuration have different physical characteristics than other cells in the expandable member. In some embodiments the expandable member can have a braided construction, and the twist region may constitute the entire group of wires, or a significant portion (e.g., more than half), of the braided wires. Such a twisted braid construction may be accomplished, for example, during the braiding process, such as by twisting the mandrel that the wires are braided onto as the mandrel is pulled along, especially along the length of the largest-diameter portion of the braided structure. The construction could also be accomplished during a second operation of the construction process, such as mechanically twisting a braided structure prior to heat-setting the wound profile over a shaped mandrel.

Any of the conduits herein act to, are configured to, and are made of material(s) that create a fluid lumen therein between an first end (e.g., distal end) and a second end (e.g., proximal end). Fluid flows into the inflow region, through the fluid lumen, and then out of an outflow region. Flow into the inflow region may be labeled herein as “I,” and flow out at the outflow region may be labeled “O.” Any of the conduits herein can be impermeable. Any of the conduits herein can alternatively be semipermeable. Any of the conduits herein may also be porous, but will still define a fluid lumen therethrough. In some embodiments the conduit is a membrane, or other relatively thin layered member. Any of the conduits herein, unless indicated to the contrary, can be secured to an expandable member such that the conduit, where is it secured, can be radially inside and/or outside of the expandable member. For example, a conduit can extend radially within the expandable member so that inner surface of the conduit is radially within the expandable member where it is secured to the expandable member.

Any of the expandable member(s) herein can be constructed of a variety of materials and in a variety of ways. For example, the expandable member may have a braided construction, or it can be formed by laser machining. The material can be deformable, such as nitinol. The expandable member can be self-expanding or can be adapted to be at least partially actively expanded.

In some embodiments, the expandable member is adapted to self-expand when released from within a containing tubular member such as a delivery catheter, a guide catheter or an access sheath. In some alternative embodiments, the expandable member is adapted to expand by active expansion, such as action of a pull-rod that moves at least one of the distal end and the proximal end of the expandable member toward each other. In alternative embodiments, the deployed configuration can be influenced by the configuration of one or more expandable structures. In some embodiments, the one or more expandable members can deployed, at least in part, through the influence of blood flowing through the conduit. Any combination of the above mechanisms of expansion may be used.

The blood pumps and fluid movement devices, system and methods herein can be used and positioned in a variety of locations within a body. While specific examples may be provided herein, it is understood that that the working portions can be positioned in different regions of a body than those specifically described herein.

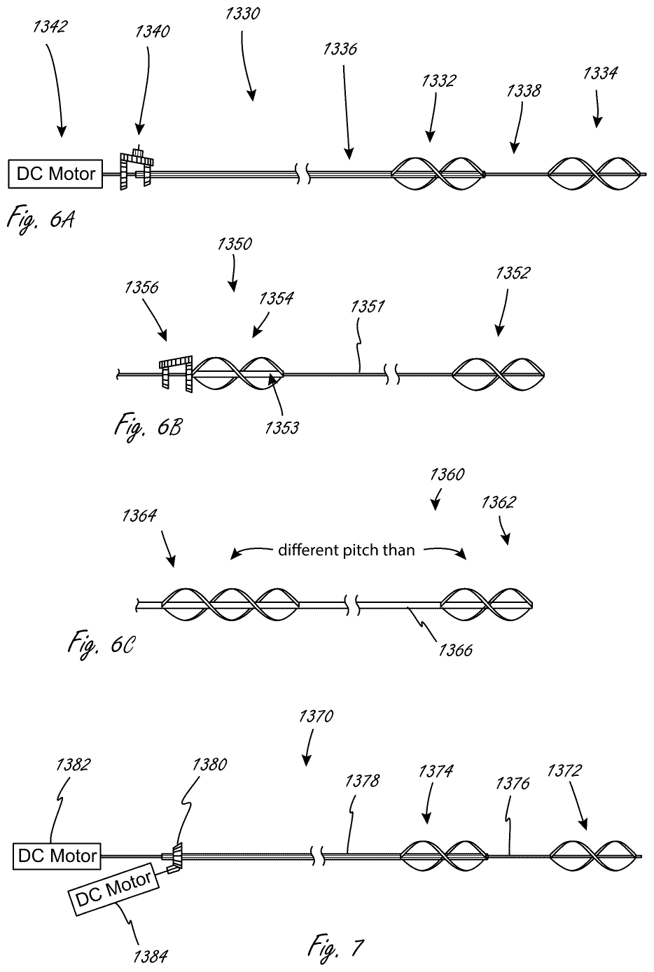

In any of the embodiments herein in which the medical device includes a plurality of impellers, the device can be adapted such that the impellers rotate at different speeds. FIG. 6A illustrates a medical device that includes gearset 1340 coupled to both inner drive member 1338 and outer drive member 1336, which are in operable communication with distal impeller 1334 and proximal impeller 1332, respectively. The device also includes motor 1342, which drives the rotation of inner drive member 1338. Inner drive member 1338 extends through outer drive member 1336. Activation of the motor 1332 causes the two impellers to rotate at different speeds due to an underdrive or overdrive ratio. Gearset 1340 can be adapted to drive either the proximal or distal impeller faster than the other. Any of the devices herein can include any of the gearsets herein to drive the impellers at different speeds.

FIG. 6B illustrates a portion of an alternative embodiment of a dual impeller device (1350) that is also adapted such that the different impellers rotate at different speeds. Gearset 1356 is coupled to both inner drive member 1351 and outer drive member 1353, which are coupled to distal impeller 1352 and proximal impeller 1354, respectively. The device also includes a motor like in FIG. 6A. FIGS. 6A and 6B illustrate how a gearset can be adapted to drive the proximal impeller slower or faster than the distal impeller.

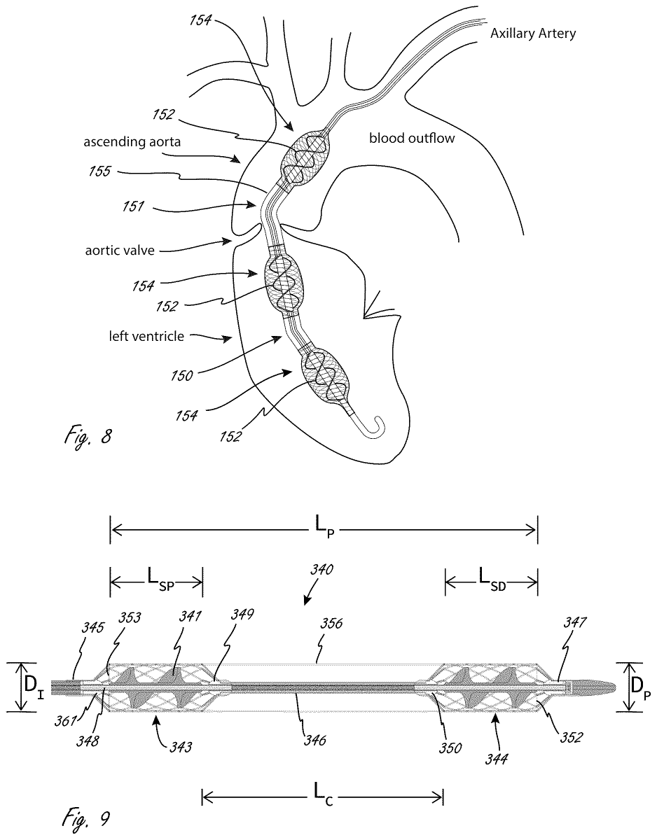

FIG. 7 shows an exemplary alternative embodiment of fluid pump 1370 that can rotate first and second impellers at different speeds. First motor 1382 drives cable 1376, which is coupled to distal impeller 1372, while second motor 1384 drives outer drive member 1378 (via gearset 1380), which is coupled to proximal impeller 1374. Drive cable 1376 extends through outer drive member 1378. The motors can be individually controlled and operated, and thus the speeds of the two impellers can be controlled separately. This system setup can be used with any system herein that includes a plurality of impellers.

In some embodiments, a common drive cable or shaft can drive the rotation of two (or more) impellers, but the blade pitch of the two impellers (angle of rotational curvature) can be different, with the distal or proximal impeller having a steeper or more gradual angle than the other impeller. This can produce a similar effect to having a gearset. FIG. 6C shows a portion of a medical device (1360) that includes common drive cable 1366 coupled to proximal impeller 1364 and distal impeller 1362, and to a motor not shown. The proximal impellers herein can have a greater or less pitch than the distal impellers herein. Any of the working portions (or distal portions) herein with a plurality of impellers can be modified to include first and second impellers with different pitches.

In any of the embodiments herein, the pump portion can have a compliant or semi-compliant (referred to generally together as “compliant”) exterior structure. In various embodiments, the compliant portion is pliable. In various embodiments, the compliant portion deforms only partially under pressure. For example, the central portion of the pump may be formed of a compliant exterior structure such that it deforms in response to forces of the valve. In this manner the exterior forces of the pump on the valve leaflets are reduced. This can help prevent damage to the valve at the location where it spans the valve.

FIG. 8 illustrates an exemplary embodiment of a pump portion that includes first, second and third axially spaced impellers 152, each of which is disposed within an expandable member 154. Conduit 155 can extend along the length of the pump portion, as in described in various embodiments herein, which can help create and define the fluid lumen. In alternative embodiments, however, the first, second, and third impellers may be disposed within a single expandable member, similar to that shown in FIG. 1. In FIG. 8, a fluid lumen extends from a distal end to a proximal end, features of which are described elsewhere herein. The embodiment in FIG. 8 can include any other suitable feature, including methods of use, described herein.

The embodiment in FIG. 8 is also an example of an outer housing having at least one bend formed therein between a proximal impeller distal end and a distal impeller proximal end, such that a distal region of the housing distal to the bend is not axially aligned with a proximal region of the housing proximal to the bend along an axis. In this embodiment there are two bends 150 and 151 formed in the housing, each one between two adjacent impellers.

In a method of use, a bend formed in a housing can be positioned to span a valve, such as the aortic valve shown in FIG. 8. In this method of placement, a central impeller and distal-most impeller are positioned in the left ventricle, and a proximal-most impeller is positioned in the ascending aorta. Bend 151 is positioned just downstream to the aortic valve.

A bend such as bend 150 or 151 can be incorporated into any of the embodiments or designs herein. The bend may be a preformed angle or may be adjustable in situ.

In any of the embodiments herein, unless indicated to the contrary, the outer housing can have a substantially uniform diameter along its length.

In FIG. 8, the pump is positioned via the axillary artery, which is an exemplary method of accessing the aortic valve, and which allows the patient to walk and be active with less interruption. Any of the devices herein can be positioned via the axillary artery. One will appreciate from the description herein, however, that the pump may be introduced and tracked into position in various manner including a femoral approach over the aortic arch.

One aspect of the disclosure is an intravascular blood pump that includes a distal impeller axially spaced from a proximal impeller. In one embodiment, the distal and proximal impellers are separated from each other. For example, the distal and proximal impellers may be connected solely by their individual attachment to a common driveshaft. This is distinct from an impeller having multiple blade rows. A distal impeller as that phrase is used herein does not necessarily mean a distal-most impeller of the pump, but can refer generally to an impeller that is positioned further distally than a proximal impeller, even if there is an additional impeller than is disposed further distally than the distal impeller. Similarly, a proximal impeller as that phrase is used herein does not necessarily mean a proximal-most impeller of the pump, but can refer generally to an impeller that is positioned further proximally than a proximal impeller, even if there is an additional impeller than is disposed further proximally than the proximal impeller. Axial spacing (or some derivative thereof) refers to spacing along the length of a pump portion, such as along a longitudinal axis of the pump portion, even if there is a bend in the pump portion. In various embodiments, each of the proximal and distal impellers are positioned within respective housings and configured to maintain a precise, consistent tip gap, and the span between the impellers has a relatively more flexible (or completely flexible) fluid lumen. For example, each of the impellers may be positioned within a respective housing having relatively rigid outer wall to resist radial collapse. The sections between the impellers may be relatively rigid, in some embodiments the section is held open primarily by the fluid pressure within.

Although not required for the embodiments therein, there may be advantages to having a minimum axial spacing between a proximal impeller and a distal impeller. For example, a pump portion may be delivered to a target location through parts of the anatomy that have relatively tight bends, such as, for example, an aorta, and down into the aortic valve. For example, a pump portion may be delivered through a femoral artery access and to an aortic valve. It can be advantageous to have a system that is easier to bend so that it is easier to deliver the system through the bend(s) in the anatomy. Some designs where multiple impellers are quite close to each other may make the system, along the length that spans the multiple impellers, relatively stiff along that entire length that spans the multiple impellers. Spacing the impellers apart axially, and optionally providing a relatively flexible region in between the impellers, can create a part of the system that is more flexible, is easier to bend, and can be advanced through the bends more easily and more safely. An additional exemplary advantage is that the axial spacing can allow for a relatively more compliant region between the impellers, which can be positioned at, for example, the location of a valve (e.g., an aortic valve). Furthermore, there are other potential advantages and functional differences between the various embodiments herein and typical multistage pumps. A typical multistage pump includes rows of blades (sometimes referred to as impellers) in close functional spacing such that the rows of blades act together as a synchronized stage. One will appreciate that the flow may separate as it passes through the distal impeller. In various embodiments as described herein, distal and proximal impellers can be spaced sufficiently apart such that the flow separation from the distal impeller is substantially reduced (i.e., increased flow reattachment) and the localized turbulent flow is dissipated before the flow enters the proximal impeller.

In any of the embodiments or in any part of the description herein that include a distal impeller and a proximal impeller, the axial spacing between a distal end of the proximal impeller and a proximal end of the distal impeller can be from 1.5 cm to 25 cm (inclusive) along a longitudinal axis of the pump portion, or along a longitudinal axis of a housing portion that includes a fluid lumen. The distance may be measured when the pump portion, including any impellers, is in an expanded configuration. This exemplary range can provide the exemplary flexibility benefits described herein as the pump portion is delivered through curved portions of the anatomy, such as, for example, an aortic valve via an aorta. FIG. 9 (shown outside a patient in an expanded configuration) illustrates length Lc, which illustrates an axial spacing between impellers, and in some embodiments may be from 1.5 cm to 25 cm as set forth herein. In embodiments in which there may be more than two impellers, any two adjacent impellers (i.e., impellers that do not have any other rotating impeller in between them) may be spaced axially by any of the axial spacing distances described herein.

While some embodiments include a proximal impeller distal end that is axially spaced 1.5 cm to 25 cm from a distal impeller proximal end along an axis, the disclosure herein also includes any axial spacings that are subranges within that general range of 1.5 cm to 25 cm. That is, the disclosure includes all ranges that have any lower limit from 1.5 and above in that range, and all subranges that have any upper limit from 25 cm and below. The examples below provide exemplary subranges. In some embodiments, a proximal impeller distal end is axially spaced 1.5 cm to 20 cm from a distal impeller proximal end along an axis, 1.5 cm to 15 cm, 1.5 cm to 10 cm, 1.5 cm to 7.5 cm, 1.5 cm to 6 cm, 1.5 cm to 4.5 cm, 1.5 cm to 3 cm. In some embodiments the axial spacing is 2 cm to 20 cm, 2 cm to 15 cm, 2 cm to 12 cm, 2 cm to 10 cm, 2 cm to 7.5 cm, 2 cm to 6 cm, 2 cm to 4.5 cm, 2 cm to 3 cm. In some embodiments the axial spacing is 2.5 cm to 15 cm, 2.5 cm to 12.5 cm, 2.5 cm to 10 cm, 2.5 cm to 7.5 cm, or 2.5 cm to 5 cm (e.g., 3 cm). In some embodiments the axial spacing is 3 cm to 20 cm, 3 cm to 15 cm, 3 cm to 10 cm, 3 cm to 7.5 cm, 3 cm to 6 cm, or 3 cm to 4.5 cm. In some embodiments the axial spacing is 4 cm to 20 cm, 4 cm to 15 cm, 4 cm to 10 cm, 4 cm to 7.5 cm, 4 cm to 6 cm, or 4 cm to 4.5 cm. In some embodiments the axial spacing is 5 cm to 20 cm, 5 cm to 15 cm, 5 cm to 10 cm, 5 cm to 7.5 cm, or 5 cm to 6 cm. In some embodiments the axial spacing is 6 cm to 20 cm, 6 cm to 15 cm, 6 cm to 10 cm, or 6 cm to 7.5 cm. In some embodiments the axial spacing is 7 cm to 20 cm, 7 cm to 15 cm, or 7 cm to 10 cm. In some embodiments the axial spacing is 8 cm to 20 cm, 8 cm to 15 cm, or 8 cm to 10 cm. In some embodiments the axial spacing is 9 cm to 20 cm, 9 cm to 15 cm, or 9 cm to 10 cm. In various embodiments, the fluid lumen between the impellers is relatively unsupported.

In any of the embodiments herein the one or more impellers may have a length, as measured axially between an impeller distal end and an impeller proximal end (shown as “L_{SD}” and “L_{SP}”, respectively, in FIG. 9), from 0.5 cm to 10 cm, or any subrange thereof. The examples below provides exemplary subranges. In some embodiments the impeller axial length is from 0.5 cm to 7.5 cm, from 0.5 cm to 5 cm, from 0.5 cm to 4 cm, from 0.5 cm to 3 cm, from 0.5 cm to 2, or from 0.5 cm to 1.5 cm. In some embodiments the impeller axial length is from 0.8 cm to 7.5 cm, from 0.8 cm to 5 cm, from 0.8 cm to 4 cm, from 0.8 cm to 3 cm, from 0.8 cm to 2 cm, or from 0.8 cm to 1.5 cm. In some embodiments the impeller axial length is from 1 cm to 7.5 cm, from 1 cm to 5 cm, from 1 cm to 4 cm, from 1 cm to 3 cm, from 1 cm to 2 cm, or from 1 cm to 1.5 cm. In some embodiments the impeller axial length is from 1.2 cm to 7.5 cm, from 1.2 cm to 5 cm, from 1.2 cm to 4 cm, from 1.2 cm to 3 cm, from 1.2 to 2 cm, or from 1.2 cm to 1.5 cm. In some embodiments the impeller axial length is from 1.5 cm to 7.5 cm, from 1.5 cm to 5 cm, from 1.5 cm to 4 cm, from 1.5 cm to 3 cm, or from 1.5 cm to 2 cm. In some embodiments the impeller axial length is from 2 cm to 7.5 cm, from 2 cm to 5 cm, from 2 cm to 4 cm, or from 2 cm to 3 cm. In some embodiments the impeller axial length is from 3 cm to 7.5 cm, from 3 cm to 5 cm, or from 3 cm to 4 cm. In some embodiments the impeller axial length is from 4 cm to 7.5 cm, or from 4 cm to 5 cm.

In any of the embodiments herein the fluid lumen can have a length from a distal end to a proximal end, shown as length Lp in FIG. 9. In some embodiments the fluid lumen length Lp is from 4 cm to 40 cm, or any subrange therein. For example, in some embodiments the length Lp can be from 4 cm to 30 cm, from 4 cm to 20 cm, from 4 cm to 18 cm, from 4 cm to 16 cm, from 4 cm to 14 cm, from 4 cm to 12 cm, from 4 cm to 10 cm, from 4 cm to 8 cm, from 4 cm to 6 cm.

In any of the embodiments herein the housing can have a deployed diameter, at least the location of an impeller (and optionally at a location between impellers), shown as dimension Dp in FIG. 9. In some embodiments Dp can be from 0.3 cm to 1.5 cm, or any subrange therein. For example, Dp may be from 0.4 cm to 1.4 cm, from 0.4 cm to 1.2 cm, from 0.4 cm to 1.0 cm, from 0.4 cm to 0.8 cm, or from 0.4 cm to 0.6 cm. In some embodiments, Dp may be from 0.5 cm to 1.4 cm, from 0.5 cm to 1.2 cm, from 0.5 cm to 1.0 cm, from 0.5 cm to 0.8 cm, or from 0.5 cm to 0.6 cm. In some embodiments Dp may be from 0.6 cm to 1.4 cm, from 0.6 cm to 1.2 cm, from 0.6 cm to 1.0 cm, or from 0.6 cm to 0.8 cm. In some embodiments Dp may be from 0.7 cm to 1.4 cm, from 0.7 cm to 1.2 cm, from 0.7 cm to 1.0 cm, or from 0.7 cm to 0.8 cm.

In any of the embodiments herein an impeller can have a deployed diameter, shown as dimension Di in FIG. 9. In some embodiments Di can be from 1 mm-30 mm, or any subrange therein. For example, in some embodiments Di may be from 1 mm-15 mm, from 2 mm-12 mm, from 2.5 mm-10 mm, or 3 mm-8 mm.

In any of the embodiments herein, a tip gap exists between an impeller outer diameter and a fluid lumen inner diameter. In some embodiments the tip gap can be from 0.01 mm-1 mm, such as 0.05 mm to 0.8 mm, or such as 0.1 mm-0.5 mm.

In any of the embodiments herein, at least one of a flow diffuser or diffusers and a stator or stators is/are located between two or more impellers along the catheter shaft, any one of which can increase fluid pressure between impellers, reduce swirl of the fluid, and/or increase the efficiency of the multiple impellers as a group.

In any of the embodiments herein, features at the fluid exit of an expandable shroud basket or expandable member are shaped to act as a flow diffuser, such as stent-like struts at the attachments between the catheter shaft outer dimension and the expandable member outer dimension, which can be blade-shaped with a twist directed to change the flow direction of blood. In any of the embodiments herein, one or more portions of the catheter shaft downstream of an impeller may flare to a larger diameter to change the angle of blood flow and cause deceleration of the blood flow to a speed closer to native aortic blood flow. Exemplary locations for a larger diameter downstream of an impeller would be at or near the area where an expandable shroud basket attaches to the catheter shaft, and/or at a bearing housing adjacent the impeller, or on or adjacent an internal motor.

In some embodiments, the pump portion can include one or more central members disposed axially in between proximal and distal impellers. The one or more central members may be coupled directly to one another, or they may not. The one or more central members may provide one or more of the following exemplary functions: structural support, flow modification, and maintaining impeller alignment. If the one or more central members provide structural support, the one or more central members may provide structural support to the outer conduit and/or to one or more impellers. For example, they may help maintain tip gap in one or more impellers. In the description that follows, the one or more central members are not in rotational operation with an impeller, unless indicated to the contrary. As used herein, the term “central member” or derivatives thereof does not imply that the member is located at at least a midpoint between two impellers, but simply that the central member is somewhere axially between the two impellers. “Central member” may thus be used interchangeably herein with the term “intermediate member.”

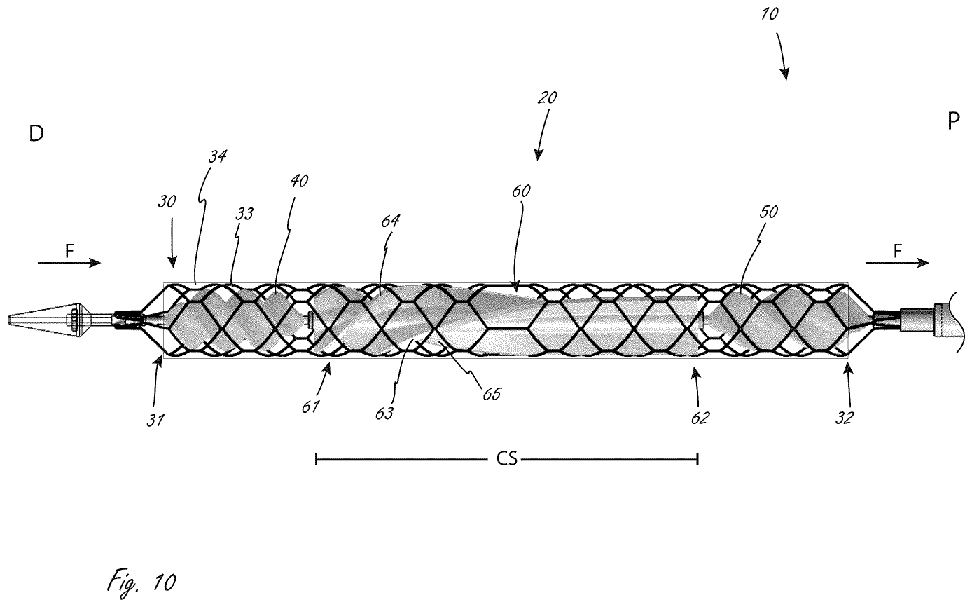

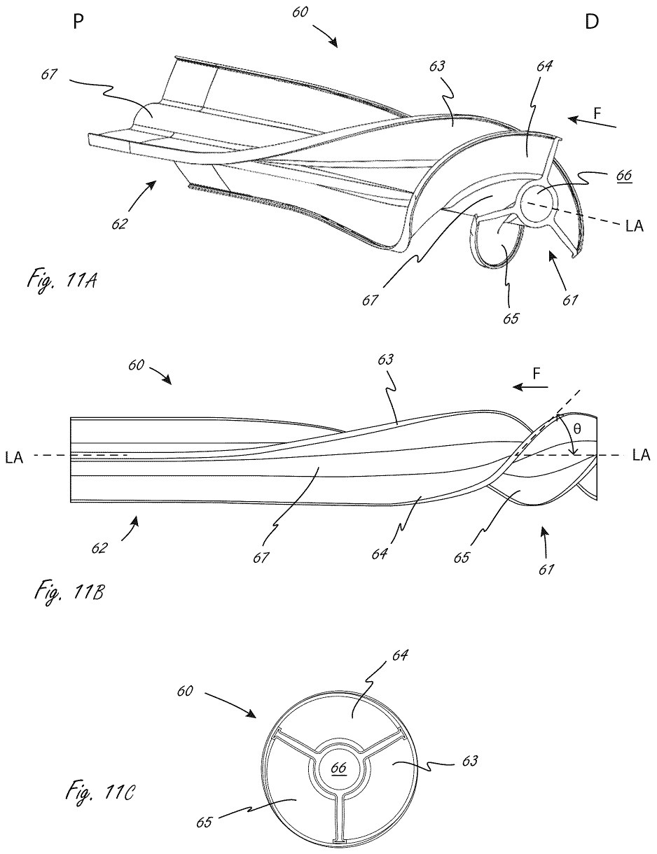

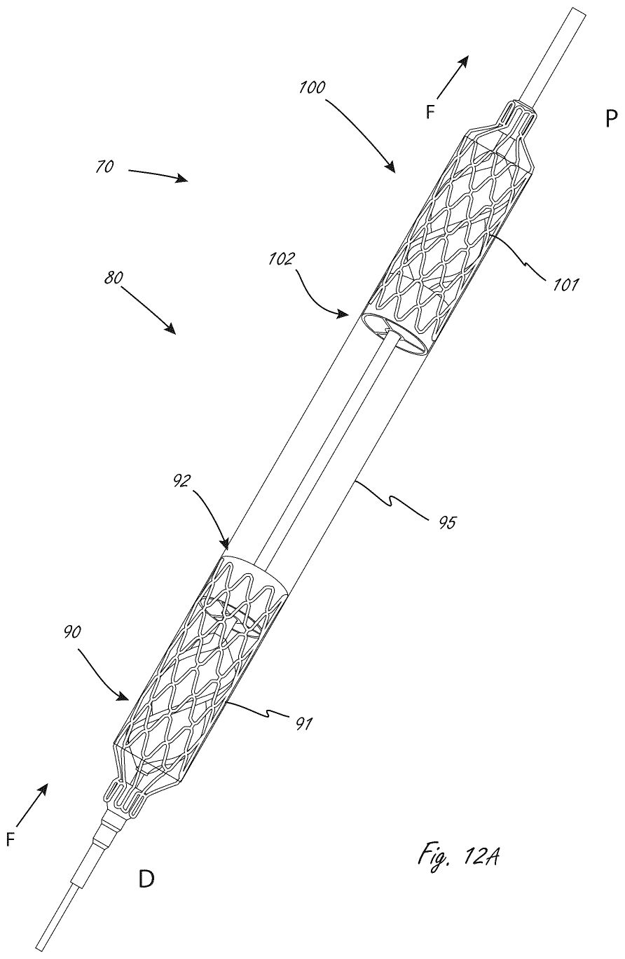

FIG. 10 is a side view illustrating an exemplary pump portion 20 of a fluid pumping apparatus 10. The distal direction is indicated with a “D” and the proximal direction is indicated with a “P.” Pump portion 20 (and other pump portions herein) may also be referred to as a distal portion herein. Pump portion 20 includes a fluid flow conduit as is described herein, as well as expandable member 30. Pump portion 20 also includes support structure 33 (which may be referred to herein as a scaffold), which in this embodiment is a stent-like member, but can be constructed using any of the examples provided herein. The conduit includes a membrane 34 that has a distal end 31 and proximal end 32. Membrane 34 is coupled to support structure 33. Membrane 34 at least partially creates and defines an internal lumen through which fluid flows when impellers 40 and 50 are activated. Membrane 34 can have any of the properties of any of the conduits that are described herein. When support structure 33 expands to the deployed and expanded configuration shown in FIG. 10, the conduit also assumes the open configuration shown in FIG. 10. Fluid flow is indicated generally in the direction of arrows “F” when impellers 40 and 50 are activated. Impellers 40 and 50 can be any of the impellers described herein and can have any of the properties described herein.

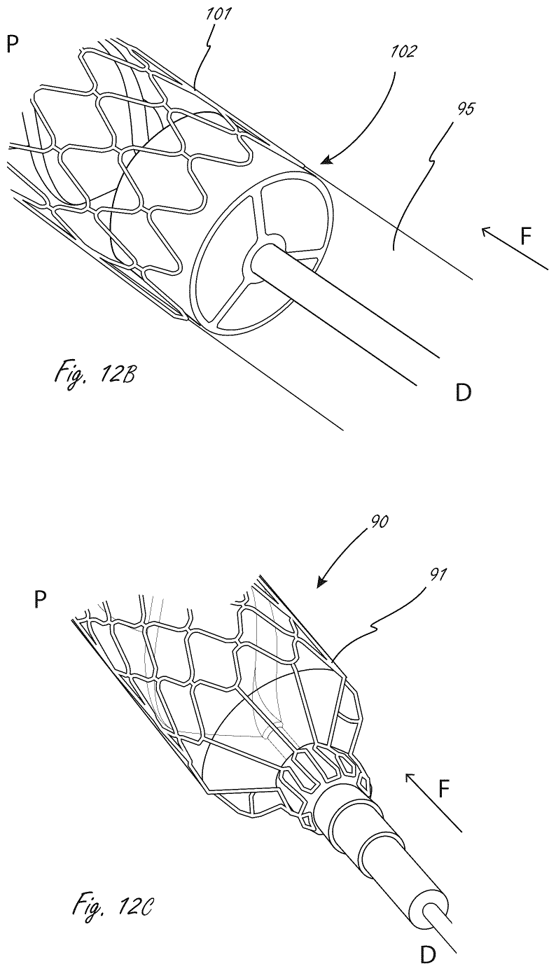

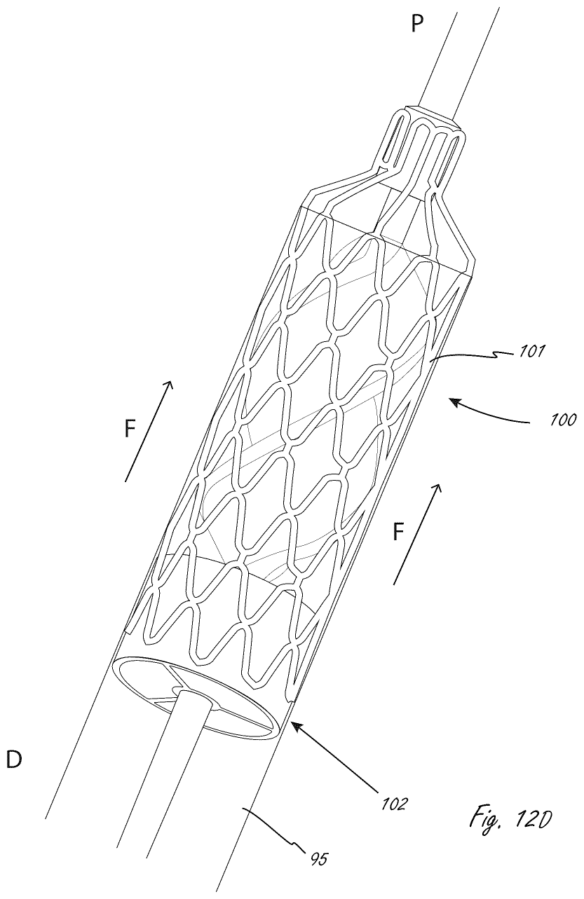

In this embodiment, pump portion 20 includes a central member 60 that is disposed axially between distal impeller 40 and proximal impeller 50. In this embodiment, central member 60 functions at least as a flow control member to modify or control the flow of blood within the fluid lumen. FIGS. 11A, 11B and 11C show perspective, side, and distal end views of central member 60, with the other pump components of the distal portion removed for clarity. Central member 60 includes hub 67 and a plurality of blades extending therefrom, in this embodiment three (i.e., blades 63, 64 and 65), although more or fewer blades may be used. Central member 60 has a distal, or front, region 61 and a back, or proximal, region 62. The blades in distal region 61 are configured to recover pressure from distal impeller 40, while the blades in proximal region 62 are configured to direct flow to proximal impeller 50. The blades in distal region 61 have a higher degree of curvature, relative to the hub, than do the blades in proximal region 62. The degree of curvature generally decreases from the distal end to the proximal end. This creates the transition between the distal region that acts more as a diffuser, to the proximal end, which acts more as a stator or guide vane. Proximal region 62 provides the functionality of a stator in this embodiment.

In addition to controlling flow and creating particular types of flow along its length, central member 60 also imparts structural support to the conduit. Member 60 provides stability in the region that is axially between impellers 40 and 50. The central region in between the impellers may receive a variety of forces thereon, and member 60 can reinforce the central region in response to those forces. For example, distal region 20 may be positioned in a heart, and in particular, the central region between the impellers may be positioned across a heart valve (e.g., aortic valve) where a great deal of motion occurs as valves open and close. The forces from valve coaptation can impart radially inward forces on expandable member 30, and member 60 can reinforce at least some portion of (including substantially the entire portion) the central span of expandable member 30 and keep the lumen open. Additionally, for example, member 60 can also reduce vibrations between the impellers that occur as the impellers rotate. The structural support provided in the central region can help maintain the gap between the tips of the impellers and the expandable member 30. Central member 60 is adapted and configured to be collapsed to a delivery configuration (like the impellers), and when expanded to the state shown in FIG. 10, it engages the expandable member 30 and provides structural support from within the lumen. In alternative embodiments, the central member 60 can be permanently attached to the expandable member, so that they collapse and expand as a unit. In this embodiment, central member 60 is thus both adapted and configured to control and create particular flow along its length in between the impellers, as well as provide structural support to the expandable member.

Any central member (e.g., control member 60) can extend axially almost the entire central span (“CS”) between the impellers. The length of central span “CS” is shown in FIG. 10. As discussed above, there are structural support advantages to have a structural support member (e.g., member 60) disposed directly adjacent an impeller, as is the case with both impellers in the embodiment in FIG. 10. If it is desired to have a single central member extend from a distal end to a proximal end (as in the embodiment in FIG. 10), then the central member can extend at least 75% of the central span distance between the impellers, or at least 80%, or at least 85%, or at least 90%, or at least 95%. It is desirable to have a spacing between the impeller and control member, which prevent friction from contact between the rotating impeller and the control member.

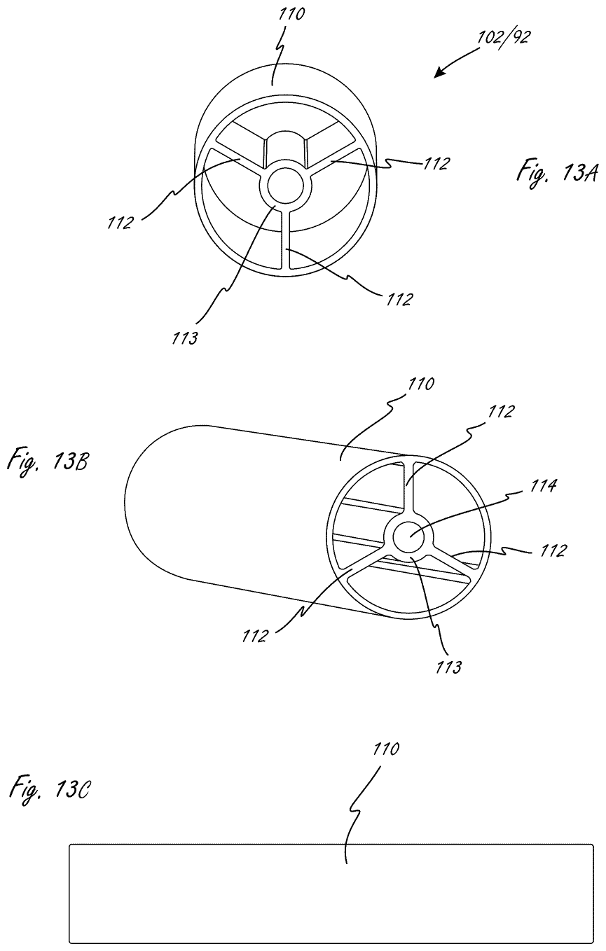

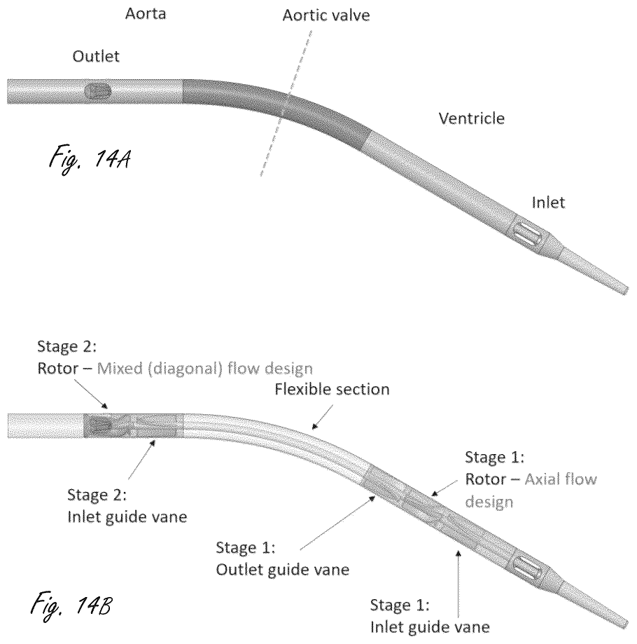

In other embodiments herein, the intermediate member may not extend along a substantial portion of the length between the impellers (see, for example, the intermediate members 92 and 102 in FIG. 12A). In any of the embodiments herein, the central member may extend no more than 5% of the central span between first and second impellers, or no more than 10%, or no more than 15%, or no more than 20%, or no more than 25%, or no more than 30%, or no more than 35%, or no more than 40%, or no more than 45%, or no more than 50%, or no more than 55%, or no more than 60%, or no more than 65%, or no more than 70%, or no more than 75%, or no more than 80%, or no more than 85%, or no more than 90%, or no more than 95%, or no more than 99%.

In any of the embodiments herein that includes multiple impellers, the axial spacing between impellers (along the length of the pump portion, even if there is a bend in the pump portion) can be from 2 mm to 100 mm, or any combination of upper and lower limits inclusive of 5 and 100 mm (e.g., from 10 mm-80 mm, from 15 mm-70 mm, from 20 mm-50mm, 2 mm-45 mm, etc.).

The length of one or more central members can be any desired length between first and second impellers.

In some embodiments, at least 50% of the central member, by length, provides functionality that is considered more like a stator than a diffuser. For example only, in the embodiment in FIG. 10, the proximal half of central member 60 functions more as a stator than a diffuser. The diffuser section may have a length sufficient to recover at least 5-50% of pressure from kinetic energy created by the preceding stage (e.g., a distal impeller). The stator section can have sufficient length to direct the flow to the next stage (e.g., a proximal impeller) without creating excessive frictional losses.

While the embodiment in FIGS. 10-11C illustrates a single central member 60 in between the impellers, in some alternative embodiments the distal, or working, portion can include more than one discrete and axially spaced central members disposed in between the impellers. FIGS. 12A-13C illustrates such an exemplary design.

FIG. 12A is a perspective view of exemplary distal portion 80 of pumping apparatus 70. The embodiment in FIGS. 12A-13C is similar in some ways to the dual-impeller design shown herein with a plurality of expandable members (e.g., FIG. 3A). One difference between FIGS. 12A-13C and FIG. 3A is that distal portion 80 includes a distal central member 92 and a proximal central member 102, each of which are disposed closely next to one of the impellers. At least a portion of central member 92 is disposed axially within the ends of structural support 91 (e.g., a stent-like device), which is a part of distal expandable member 90. Central member 92 is also radially within structural support 91. At least a portion of central member 102 is disposed axially within the ends of structural support 101 (e.g., a stent-like device), which is a part of proximal expandable member 100. Central member 102 is also radially within structural support 101. Conduit 95 (which can have any of the conduit properties described herein) extends from a distal end to a proximal end, including extending axially between central member 92 and central member 102.

FIGS. 13A, 13B and 13C show end perspective, perspective, and side views of central members 92 and 102 (the other components are not shown for clarity). The central member include an outer annular member 110 and an inner annular member 113, between which a plurality of blades 112 extend.

Similar to central member 60 in FIG. 10, central members 92 and 102 are adapted and configured to provide fluid control and to provide structural support. Central members 92 and 102 are each disposed radially within and at least partially axially within the expandable members 90 and 100. The annular member 110 and radially extending blades 112 provides radial reinforcement and support, and can thus help keep the lumen open, maintain tip gap, and reduce vibrations between the impellers. In this embodiment, the blades are also configured such that central members 92 and 102 act as stators. For example, proximal central member 102 can direct the flow before it reaches the proximal impeller. Distal central member 92 can also help to recover pressure. “Direct the flow” and derivatives thereof as used herein can include altering the ratio of axial to radial flow components. For example, any stator functionality (and any components that function a stator) herein can increase axial flow components and reduce radial flow components.

In this embodiment a portion of the central span between the impellers does not include an expandable member or a support member, but does include conduit 95 (e.g., a flexible membrane). This is similar to the embodiment in FIG. 3A. Deformable conduit 95 may allow the central region to deform to some extent where valve leaflets are coapting. Support members 92 and 102, however, help reinforce the ends of the expandable members 90 and 100 even though the conduit 95 can deform in more central regions.

An additional difference between the embodiment in FIGS. 3A and 12A-13C is that in FIGS. 12A-13C, the expandable members do not have struts on the inner portion (closer to the middle along the longitudinal axis) of the expandable members. Central members 92 and 102 replace those struts.

Central members 92 and 102 are collapsible and expandable, just as are the impellers herein. Central members 92 and 102 are secured to a component passing through lumen 114 (see FIG. 13B), and are secured so that they do not rotate when the impellers rotate. A rotatable axle passes through the central members 92 and 102. Central members 92 and 102 are directly adjacent to the impellers, but spaced just enough to prevent any friction between the parts.

In other embodiments there can be more than two central members axially spaced apart and in between the impellers. For example, one or more separate central members could be disposed between central members 92 and 102, and, for example, secured to the same elongate shaft to which central members 92 and 102 are secured.

Central members 92 and 102 can be permanently affixed to expandable member 90 and 100, respectively, such that they expand and collapse together. For example, the radially outer surface of annular section 110 can be secured to the expandable member. Alternatively, central members 92 and 102 are not affixed to the expandable members, but the central members are sized to contact/engage the expandable members when both are in the deployed configurations.

An axle that is operably connected to the impellers can extend through a shaft to which the central members 92 and 102 are secured, such that the axle can rotate within the non-rotating elongate shaft to drive the rotation of the impellers without causing rotation of the central members.

In some alternative embodiments not shown, aspects of the central members 92 and 102 can be incorporated into a single central member design. For example, the annular outer region 110 from which the blades 112 extend can also be incorporated into all or some portion of the length of a single central member. For example, in some alternative embodiments to FIG. 10, the central member 60 can include one or more annular outer regions anywhere along the length of central member 60. For example, central member 60 could include a single outer annular region that extends along its length, and blades 63, 64, and 65 can extend from the single outer annular region. Or, for example, central member 60 could include a plurality of annular outer regions disposed at any location along its length. For example, distal and proximal end regions of central member 60 can each include a discrete annular outer region from which the blades 63, 64, and 65 extend. The discrete annular outer regions can be of any desired length and occupy any desired percentage of the axial span between the impellers. Or, for example, the central member could also include a third discrete annular outer region in the center of central member 60. Additional discrete outer annular regions can be axially spaced apart along the length of central member 60 (or any other single central member).

In some alternative embodiments not shown, aspects of the central member 60 can be incorporated into a design that includes a plurality of central members (e.g., central members 92 and 102). For example, blades 112 in central members 92 and 102 (shown in FIGS. 13A and 13B) need not have straight configurations, but can be curved to some extent like portions of the blades 63, 64, and 65 shown in FIG. 10. Or, portions of the blades 112 can be straight and portions can be curved, such as are the blades 63-65 in FIG. 10. For example, distal central member 92 can have blades with curved portions, while proximal central member 102 can have straight blades. In these designs, central member 92 could act more to recover pressure, and proximal central member 102 could function more like a stator that directs flow.

Additionally, distal central member 92 need not have the same configuration as proximal central member 102.

Additionally still, in other embodiments, aspects of the distal regions 20 and 80 in FIGS. 10 and 12A can be incorporated with the other distal region. For example, in FIG. 10, support structure 33 extends across the entire central span between the impellers. In distal region 80 shown in FIG. 12A, a support structure can similarly extend across the span between central members 92 and 102. For example, distal support structure 91 could extend proximally into the central span and also form the proximal support structure 101.

The fluid pumps described with respect to FIGS. 10-13C can be positioned in any anatomical location described herein. In exemplary methods of use, the fluid pumps can be used in methods that position the devices as is shown in FIG. 4 when in use. The entirety of the description herein related to FIG. 4 is incorporated by reference for all purposes for the embodiments in FIGS. 10-13C. For example, fluid pump 10 can be placed across an aortic valve so the distal impeller is positioned in a left ventricle and the proximal impeller is positioned in an ascending aorta. In this position, the central region of the distal portion, including a portion of the control member 60, is positioned at the location of the aortic valve.

Any of the pump portions herein that include a plurality of impellers may also include more than two impellers, such as three, four, or five impellers (for example).

While some of the embodiments above describe pump portions or components that are collapsible and expandable (or at least movable between collapsed and expanded configurations), in any of those embodiments the components and expandable outer housing may also be non-expandable and non-collapsible. That is, any of the components in those embodiments may be present, but the components may be non-expandable variations of those components. For example, the impellers above may be non-expandable rather than expandable.

FIGS. 14A and 14B show side views of a distal portion of an exemplary embodiment of a blood pump in which the components are not expandable and collapsible. All the components in this embodiment can be rigid, fixed pieces.

FIG. 14B illustrates internal components that cannot be seen in FIG. 14A. The descriptions in FIGS. 14A and 14B are illustrative and not limiting. The pump portion shown in FIGS. 14A and 14B includes a Stage 1 section and a Stage 2 section, axially spaced apart along the length of the pump portion. In this embodiment the central section between the two stages (labeled generally as “Flexible section”) has a bend formed therein, which may extend along any portion of the central section between the stages, such as at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. The bend can be manufactured into the central region so that extracorporally the bend is present, but the central region can be flexible enough so that it can reconfigured to a straightened delivery configuration within a delivery device such as a delivery sheath or introducer.

The pump portion includes a plurality of axially aligned inlet apertures distal to the Stage 1 components. There are two inlet apertures in FIGS. 14A and 14B, but there may be more than two inlet apertures. There are also two outlet apertures that are axially aligned with a portion of the Stage 2 rotor. The inlet and outlet apertures extend through a radially outer wall of the pump portion. Distal is to the right in the figures, and proximal is to the left in the figures. In various embodiments the pump portion includes a set of inlet apertures distal the Stage 1 components and a set of outlet apertures proximal the Stage 2 components. In various embodiments the pump portion includes a set of inlet apertures distal the distal pump and a set of outlet apertures proximal the proximal pump. In various embodiments, no apertures (for inlet or outlet) are between the Stage 1 and Stage 2 components. In various embodiments, no apertures (for inlet or outlet) are between the distal and proximal pump impellers.

Stage 1 in this embodiment includes a distal impeller (labeled as Rotor), an inlet guide vane distal to the rotor, and an outlet guide vane proximal to the distal rotor. The vanes (and any vanes herein) are considered generally to be flow modification elements or a derivative as that term is used herein. Any of the vanes and rotors can include a hub and extending blades as shown, or can include other known impeller and stator/vane designs. The vanes (and any flow modification components herein) are positioned closely next to the distal impeller, such as less than 10 mm away (along the length of the device), or less than 9 mm away, or less than 8 mm away, or less than 7 mm away, or less than 6 mm away, or less than 5 mm away, or less than 4 mm away, or less than 3 mm away, or less than 2 mm away, or less than 1 mm away. “Closely” as used herein can include any of these axial distances. “Closely” as used herein can also refer to a distance less than two times a diameter of the central lumen.

Stage 2 in this embodiment includes a proximal impeller (rotor) and an inlet guide vane distal to the proximal impeller. All of the disclosure above related to the vanes in Stage 1 is incorporated and can be incorporated into Stage 2 vanes.

In this example, the Stage 1 (distal) rotor is configured as an axial flow impeller, and proximal impeller (Stage 2) is configured as a mixed (diagonal) flow impeller, but these are illustrative and other impeller designs can be used for either impeller.

The pump portion in this embodiment includes a flexible outer housing between the stages. The flexible outer housing can be, for example, a flexible polymeric material that is formed with a slightly degree of curvature and can be straightened for delivery, and is coupled to the distal stage and proximal stage sections. In some embodiments the flexible central section could be a very thin walled rigid material, which imparts some flexibility. In other embodiments, for example, the flexible section could include a plurality of elongate support members (e.g., nitinol wires) to which a flexible membrane is attached. The elongate support members can be formed with bends therein and spaced around the periphery of the flexible section, so that the flexible membrane forms a lumen therethrough. In some embodiments, the flexible section can include a laser cut tube (e.g., laser cut polymeric or metallic material, e.g., nitinol) with one or more slots cut out in at least a section to impart flexibility (e.g., creating a spine along one side with ribs extending around at least a portion of the periphery, the ribs formed by cutting out material), and a membrane like material can be affixed to the slotted tubular member to cover the removed material. The flexible material could also include a stent like device that is configured with a bend, and a membrane like material covering the stent apertures.

As used herein, “axially spaced” includes embodiments in which a bend exists in the outer profile (e.g., FIGS. 14A and 14B), wherein a bend can be included in any of the embodiments herein. Axially spaced as that phrase is used anywhere herein is meant to refer to spacing along the device, even if there is a bend in the outer profile of the pump portion (e.g., FIGS. 14A and 14B). It may refer to spacing along a longitudinal axis of the pump portion, for example.

In alternative embodiments to that shown in FIGS. 14A and 14B, not all components shown need to be included. For example, any of the vanes may not be present, depending on flow needs.

Any of the other disclosure herein related to any aspect of a pump device or method of use (e.g., external motors, placement when used) is incorporated by reference into the embodiments in FIGS. 14A and 14B.

The description shown in FIG. 14A illustrates an exemplary placement of the device, showing surrounding/ambient anatomy. The distal impeller can be positioned in the left ventricle while the proximal impeller is positioned in the ascending aorta, and the impellers can be spaced accordingly.

Blood pumps, such as any of the intravascular pumps herein, may benefit from having one or more fluid paths through which fluid can flow through the device. For example without limitation, blood pumps may benefit from having one or more fluid paths through which fluid can flow to perform any of these exemplary functions: cooling rotating components (e.g., a drive cable) to prevent their overheating; flushing small particulates that may break off rotating components (e.g., a drive cable) to prevent the rotating parts from being damaged by the small particulates; lubricating rotating components (e.g., one or more bearings), and preventing blood ingress into the pump (e.g., near or at a distal end of the pump). Fluid delivery through the one or more flow paths may provide any number of these functions.

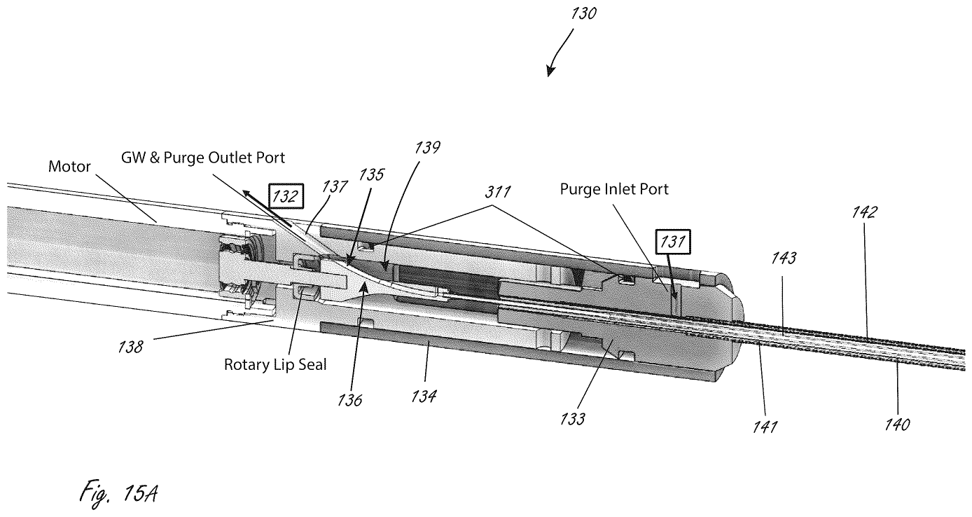

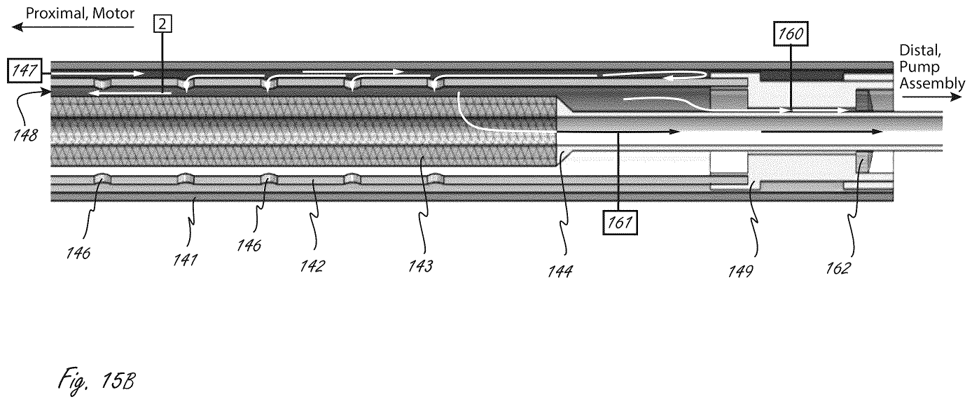

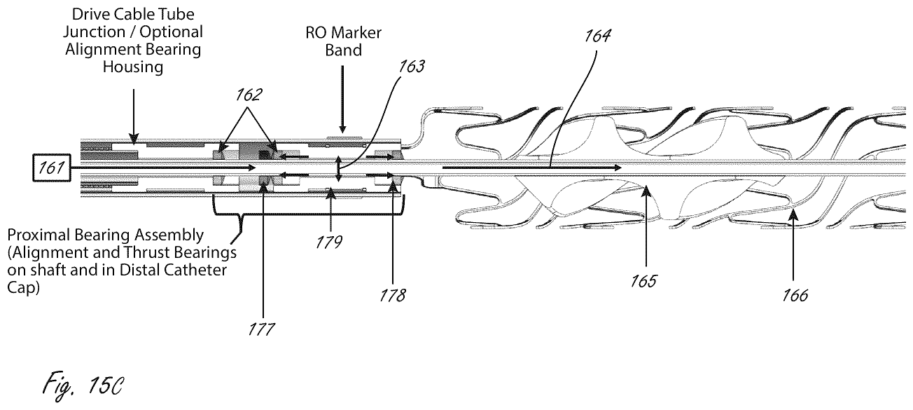

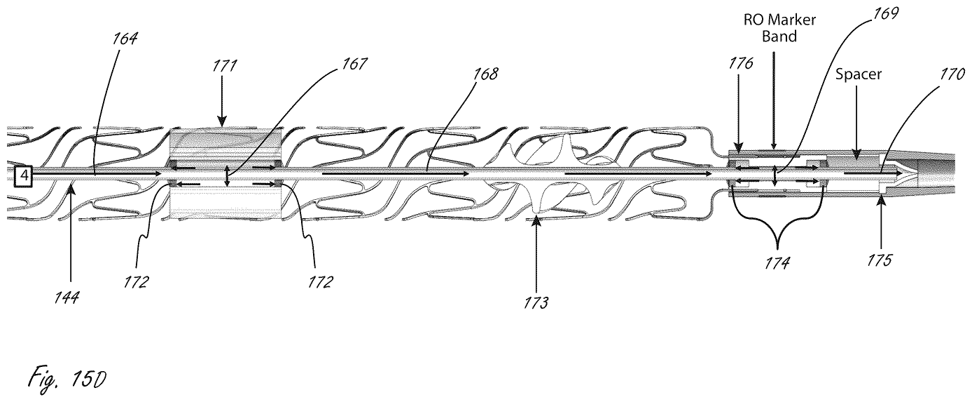

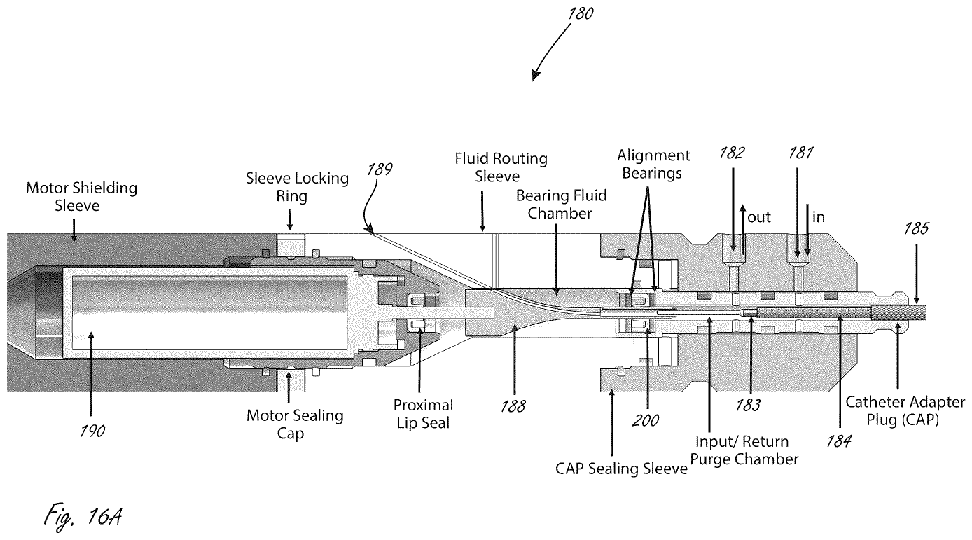

FIGS. 15A-15D illustrate an exemplary embodiment of a fluid delivery system incorporated into an exemplary fluid pump (e.g., blood pump) with a fluid inlet port and a fluid outlet port. FIG. 15A illustrates a portion of the device that is proximal to the one or more impellers, and in this embodiment includes a proximal end of a catheter, a motor assembly that causes the rotation of a drive cable and impeller(s), a fluid inlet port, and fluid outlet port, and a guidewire port that allows access to a guidewire pathway or lumen.

FIG. 15B shows a region of the device that is distal to the region shown in FIG. 15A, but includes some of the catheter components that are shown in FIG. 15A. FIG. 15C shows a region of the device distal to the region in FIG. 15B, and FIG. 15D shows a region of the device distal to the view in FIG. 15C.

While FIGS. 15A-15D illustrate different sections of an exemplary blood pumping device, it is understood that in alternative embodiments aspects of the system can vary. For example, in alternative embodiments the portion of the device with the impellers can vary and could only include a single impeller, or the expandable housing around the impeller could have a wide variety of configurations. It is understood that individual regions of the device can be incorporated by themselves into a variety of different types of blood pumps.

One aspect of this exemplary embodiment includes a guidewire access port that also functions as a fluid port, and in this embodiment a fluid outlet port. A motor sealing cap 138 includes, formed therein, a guidewire channel 137, including a guidewire port in a radially side surface that provides access from outside the device to channel 137. The motor sealing cap may be an optional component, and the guidewire channel 137 can alternatively be formed in a different part of the device (e.g., which may not function as a motor sealing cap). The device also includes drive cable coupler 135, which includes formed therein a guidewire channel 136, which is a portion of a guidewire pathway. Drive cable coupler 135 is rotated by the motor, and causes the rotation of drive cable 143, which causes rotation of the one or more impellers in the pump portion. These components are thus considered to be in rotational communication. Channel 137, including the guidewire port, is formed in the device and is not adapted to rotate when the motor rotates. Channel 136 formed in drive cable coupler 135 rotates when the drive cable coupler rotates. When drive cable coupler 135 is in the position shown in FIG. 15A, channel 137 is in alignment with channel 136, which allows a guidewire to be advanced through or removed from channel 137 and through channel 136. If the guidewire is being inserted, the guidewire can then be advanced further distally through the entire device and out a distal end, described in more detail below. As is also described in more detail below, the guidewire access port also acts as a fluid outlet port that allows return fluid to flow from return area 139 out of the outlet port.

One of the advantages of having the guidewire access port (part of channel 137) in the location that it is in this embodiment, is that, if needed after the pump portion has already been advanced to a location within the patient, a guidewire can be reinserted into the port and inserted all the way to and out of the distal end. Importantly, the guidewire can be reinserted without having to remove most of the device from the patient like with some rapid exchange designs, and without having to remove the motor assembly. This exemplary embodiment thus allows easy reentry of a guidewire without having to remove the motor assembly, and without having to remove the device from the subject.

Being able to reinsert the guidewire during use can be advantageous because it can, for example without limitation, allow for repositioning of the pump portion if desired or needed. For example, if the pump portion moves out of position relative to an anatomical landmark (e.g., an aortic valve), a guidewire may need to be inserted to safely reposition it relative to the anatomical landmark.

Because the guidewire path extends through a rotational component (e.g., drive cable coupler 135), it is important that the guidewire not be present in the guidewire path when the rotating component is active. The apparatuses herein can also include an automated sensing mechanism to detect the presence of the guidewire in the guidewire pathway, and/or a prevention mechanism that prevents the motor from being activated if the guidewire is in the lumen. For example without limitation, there could be a sensor that can selectively detect the presence of the guidewire in the guidewire pathway, and communicate that to a controller that prevents the motor from being activated.

In this embodiment there is a single fluid inlet channel or lumen 131 into which fluid can be delivered into the device. FIG. 15B illustrates a region of the device and illustrates different pathways the fluid can take after it has been delivered into the device. After the fluid is advanced into fluid inlet port channel 131 (which includes an inlet port), it travels through a space 147 between clean purge tube 141 and drive cable tube 142. This is considered clean input fluid. This pathway deadens at distal catheter cap 149. The fluid passes through the one or more apertures 146 formed in a distal region of drive cable tube 142 as shown in FIG. 15B, entering into an annular space between drive cable tube 142 and drive cable 143. Some of this fluid (optionally most of the fluid) returns in the proximal direction through this annular space, lubricating and cooling drive cable 143 and flushing potential particulate along its path. This return fluid continues to flow proximally and into area 139 shown in FIG. 15A, and continues to flow through channel 137 and out of the fluid port (which is also the guidewire access port). A fluid outlet port thus also functions as a guidewire access port in this embodiment.

While most of the fluid returns proximally to area 139, some of the fluid, after it passes through apertures 146, continues distally beyond the distal end of the drive cable 143. Some of the fluid follows proximal bearing path 160 through alignment bearing 162 to prevent blood ingress. Fluid flow along path 160 to bearing 162 can be controlled by, for example, controlling input flow pressure and throttling of the return fluid at the proximal region of the device.

Some of the fluid, after passing through apertures 146, will flow through drive cable 143, along path 161, and will continue distally through the device (e.g., through hypotube 144) and out holes to lubricate any rotating surfaces and to prevent blood ingress, described in more detail below. Guidewire lumen 145 is thus positioned to also function as a distal bearing fluid flow path.

Some fluid flows distally along path 161, as shown in FIG. 15C, and passes through holes along path 163, to lubricate one or more of bearings 162, thrust bearing 177, and alignment bearing 178. Some of the fluid continues distally in the direction of arrow 164 shown in FIG. 15C, through impeller 165 (which in this embodiment is a proximal impeller). Some of the fluid passes through apertures along path 167 to lubricate optional alignment bearings 172 that support central member 171, which may be any of the collapsible support members, including any of the central or intermediate members herein. Some fluid continues distally through the guidewire lumen in the direction of arrow 168, through optional distal impeller 173. Some fluid passes through holes along path 169 to lubricate bearings 174 that are distal to the distal impeller. Some of the fluid may also flow through valve 175 and out the distal end of the device, helping prevent blood ingress.

In this exemplary embodiment a single flow path flowing through a tubular member (path 161 that extends distally through guidewire lumen shown in FIG. 15B) leads to (is in fluid communication with) at least three distally located bearing lubricating fluid paths, 163, 167, and 169, which lubricated three axially spaced bearing regions. In some alternative embodiments, there may be a single bearing region that is lubricated, two bearing regions that are lubricated, or even more than three bearings regions that are lubricated, depending on the number of structures disposed within the expandable housing that require bearings and thus lubrication.

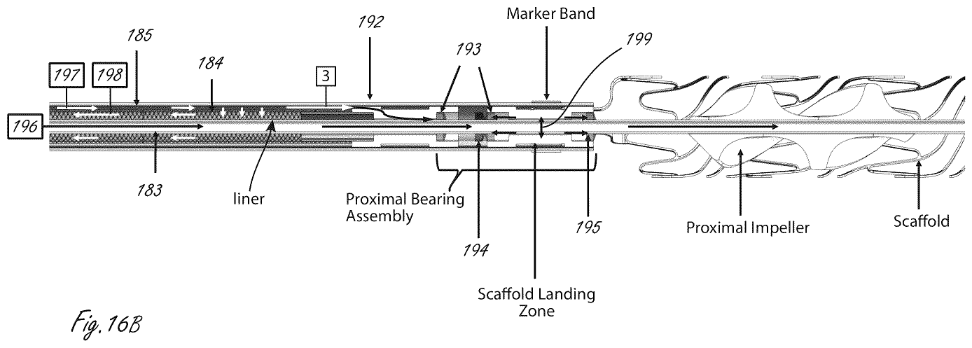

An exemplary method of using the device in FIGS. 15A-D includes inserting a guidewire near a target location (e.g., into a left ventricle via femoral artery access), then feeding the distal guidewire port over the guidewire and advancing the device over the guidewire towards the target location (e.g., an aortic valve). The method can also include removing the guidewire from the guidewire path, and coupling the proximal portion shown in FIG. 15A to a fluid inlet coupler and a fluid outlet coupler at the inlet and the outlet fluid locations, respectively. The motor can be activated to activate the one or more impellers. If the guidewire needed to be reinserted, the fluid out connector can be removed and a guidewire can be reinserted (e.g., for repositioning). The guidewire can then be removed and the fluid outlet coupler can again be put into fluid communication with the guidewire pathway. These methods or any of them individually can be incorporated into the use of any of the suitable devices herein, such as the device in FIGS. 16A and 16B. Additionally, any of the steps in any of the other exemplary methods of use herein, such as those below, may be incorporated into a use of the blood pump in this embodiment.

FIGS. 16A and 16B illustrate an exemplary embodiment of a fluid delivery system incorporated into an exemplary fluid pump (e.g., blood pump) with a first flow path with a first fluid inlet port and a first fluid outlet port. In this embodiment, however, there is also a second fluid flow path that is not in fluid communication with the first flow path. The device 180 in FIGS. 16A and 16B is similar to that shown in the embodiment in FIGS. 15A-D, except in this embodiment the fluid path 161 from FIG. 15B does not originate as fluid that flows through the drive cable. In this embodiment the fluid flow path that includes the guidewire lumen (see fluid path 196 in FIG. 16B) is in fluid communication with a separate and second fluid inlet port 189, which is also located to function as a guidewire access port, as shown in FIG. 16A. Drive cable 183 has a drive cable liner 187 on its inner surface to seal off the distal bearing flow path 196 (through the guidewire lumen). In this embodiment the guidewire access port does not function as a fluid outlet, like in FIGS. 15A-D, but as a fluid inlet port, and thus still functions as a fluid port or fluid access.

The blood pump also includes a first fluid path that includes inlet port 181 and outlet port 182 as shown in FIG. 16A. This flow path is very similar to the path in FIGS. 15A-D, except that it does not include the path through the drive cable and hypotube (i.e., does not include the guidewire lumen). The fluid is advanced through port inlet port 181, flows distally along path 197 in FIG. 16B, which is between clean purge tube 185 and drive cable tube 184. This path deadens at a distal catheter cap, just as in the embodiment in FIGS. 15A-D. The fluid flows through holes in drive cable tube 184, and returns proximally in the annular space between drive cable tube 184 and drive cable 183. In this part of the path the fluid lubricates and cools the drive cable and flushes potential particulate along its path, carrying them proximally to fluid exit port 182 shown in FIG. 16A. Seal 200 prevents fluid from passing proximally to seal.

Fluid flowing through the first fluid path thus lubricates and cools the drive cable, as well as flushes potential particulates and returns to exit port 182. Fluid flowing through the second fluid path travels further distally through the system, and lubricates one or more distal bearings, just as in the embodiment in FIGS. 15A-D. For example, path 199 shown in FIG. 16B is the same as path 163 in FIG. 15C, which lubricates bearings in that bearing region. While not shown, the fluid flow path distal to the view shown in FIG. 16B can be exactly the same as in FIG. 15D, thus lubricating additional bearings, and optionally exiting through a valve at a distal end of the device. This second flow path can thus also prevent ingress of blood, which is described more fully in FIGS. 15A-D.

In any of the devices herein, the pump portion can include a distal end valve distal to the impeller to seal off the distal guidewire port after the guidewire is removed, but allows for guidewire reinserting therethrough.

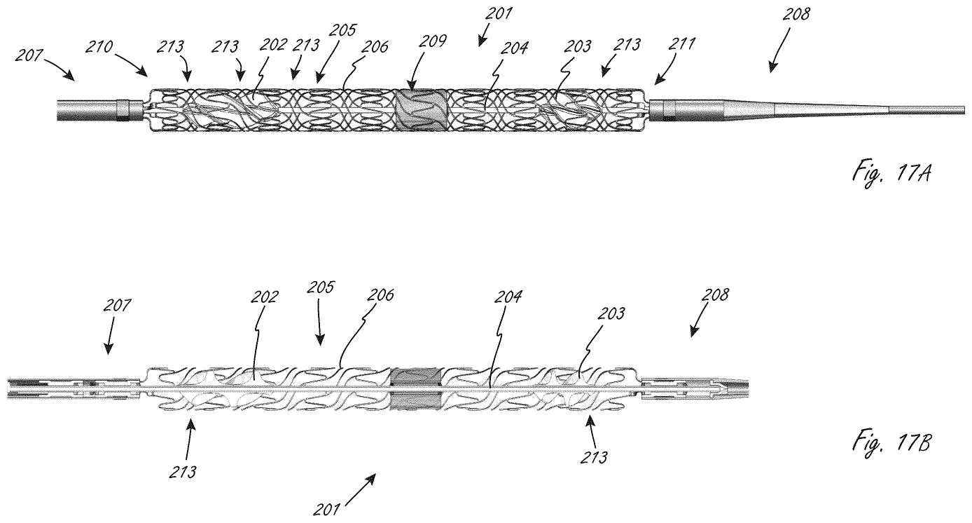

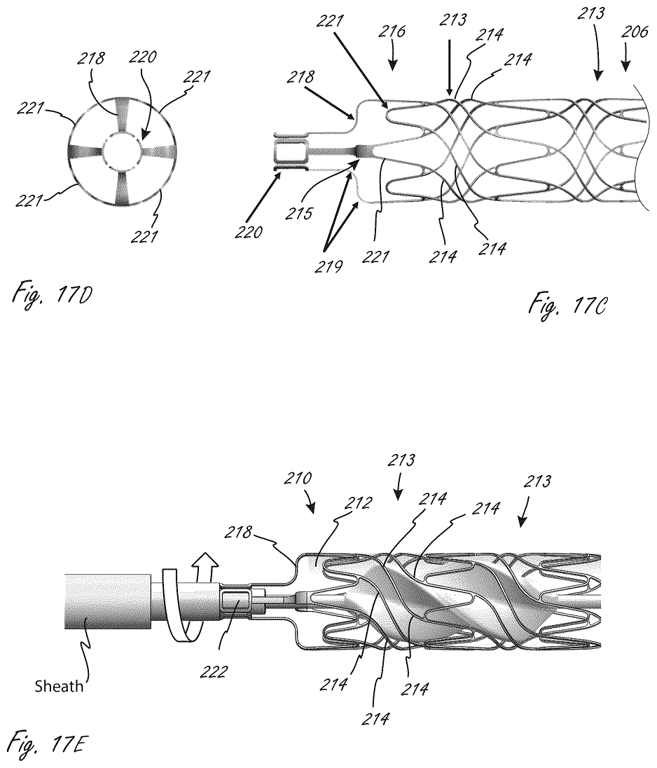

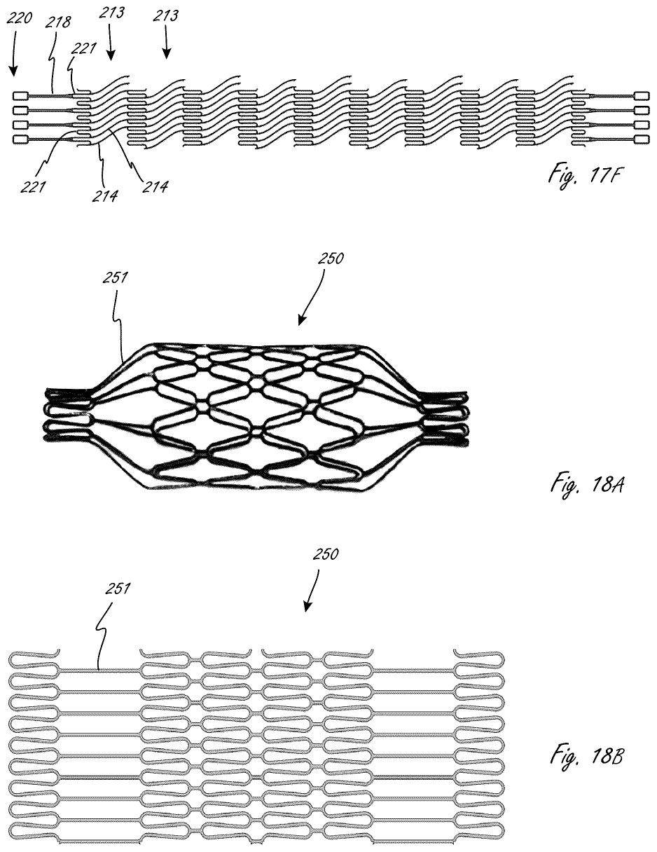

FIGS. 17A-17F illustrate exemplary pump portion 201 of an exemplary blood pump. Pump portion 201 can be used interchangeably with any other aspect of the any of the blood pumps herein. Pump portion 201 is also shown in the embodiments in FIGS. 15A-16B, and thus any features or methods of use described therein are incorporated by reference into this embodiment. Additionally, not every aspect of this embodiment needs to be included, and instead, any suitable feature(s) in pump portion 201 may be replaced with a different feature or method of use from a different suitable embodiment or part of this disclosure. For example, either impeller in pump portion 201 can be replaced with any suitable impeller from any other part of this disclosure. FIG. 17A is a side view, and FIG. 17B is a sectional side view.

Pump portion 201 includes drive cable tubular member 204, to which distal impeller 203 and proximal impeller 202 are secured. Rotation of drive cable tubular member 204, via rotation of the drive cable (not shown), causes rotation of the impellers. More or fewer than two impellers may be included in the pump portion.

Pump portion 201 also includes a collapsible housing 205, which includes collapsible support structure 206 (which may be referred to herein as a scaffold) with proximal end 210 and distal end 211, and conduit 212 (see FIG. 17E), which forms a fluid lumen between a distal end and a proximal end of the fluid lumen.

Pump portion 201 includes optional intermediate (which may be referred to herein as central, or in between impellers) member 209 between two impellers, which may be any central member or members herein.

In any of the embodiments herein, the distal impeller can have a length that is less than a proximal impeller, such as is shown in the device in FIG. 17A.

FIG. 17C is a side view of a proximal portion of support structure 206 in an expanded configuration (other parts not shown for clarity). FIG. 17D is a proximal end view of the support structure. The region shown is generally surrounding impeller 202 in FIGS. 17A and B. Support structure 206 can be formed using a variety of techniques, such as laser cutting a tubular starting material. Support structure 206 includes a plurality of arms (four in this embodiment) that, at the proximal region, transition from a larger diameter to a smaller diameter in regions 218. Each of the arms has a bend in regions 219, and is vertical in between the bend regions, as shown. The vertical region can help stabilize the transition region between the larger diameter and smaller diameter regions, and reduce and preferably eliminate the influence on the fluid at the outflow.

In the larger diameter region of the support structure, the support structure 206 includes staggered peaks 221 (only two are labeled), alternating every other peak. Staggered in this context refers to the axial location of the end of the peak. Each of the four arms forms a peak that extends further proximally than adjacent peak. The staggered peaks can facilitate sheathing and offset packing volume during collapse of the pump portion. A peak as used herein may also be considered a valley depending on the orientation, similar to how convex and concave are relative terms.

Support structure 206 also includes a plurality of hub features 220 that are each configured to stably bond to a component 222 (there are four in this embodiment) at a distal region of scaffold landing zone 179 (see FIG. 17E). Hub features 220 can constrain axial movement along bearing hubs.

Support structure 206 also includes axially spaced helical regions 213 (only some are labeled in FIGS. 17A and 17B) that include a plurality of arms (or portions of arms) that have helical configurations. In FIG. 17C, helical region 213 includes helical arms 214 (only four are labeled). In this embodiment, the helical arms extend between adjacent non-helical regions of the support structure. The regions in between the helical regions can have any number of configurations, and exemplary configurations are shown. In this exemplary embodiment, proximal impeller 202 axially overlaps with a least a portion of two adjacent helical regions 213, and distal impeller axially overlaps with at least a portion of two adjacent helical regions 213. Any impeller can axially overlap with one or more helical section 213. The pitch of the helical arms can vary.

FIG. 17F illustrates the scaffold design from FIGS. 17A-E in a flattened planar view to further illustrate the scaffold pattern. Only some of the portions of the scaffold are labeled for clarity.

The axial length of one or more helical regions 213 can be shorter than an axial length of an impeller with which it axially overlaps. For example, in FIG. 17E, the helical regions 213 are each shorter than the overall length of the impeller. The axial length of the helical regions 213 is also less than the length of distal impeller 203, even if a single helical region does not completely axially overlap with the impeller.

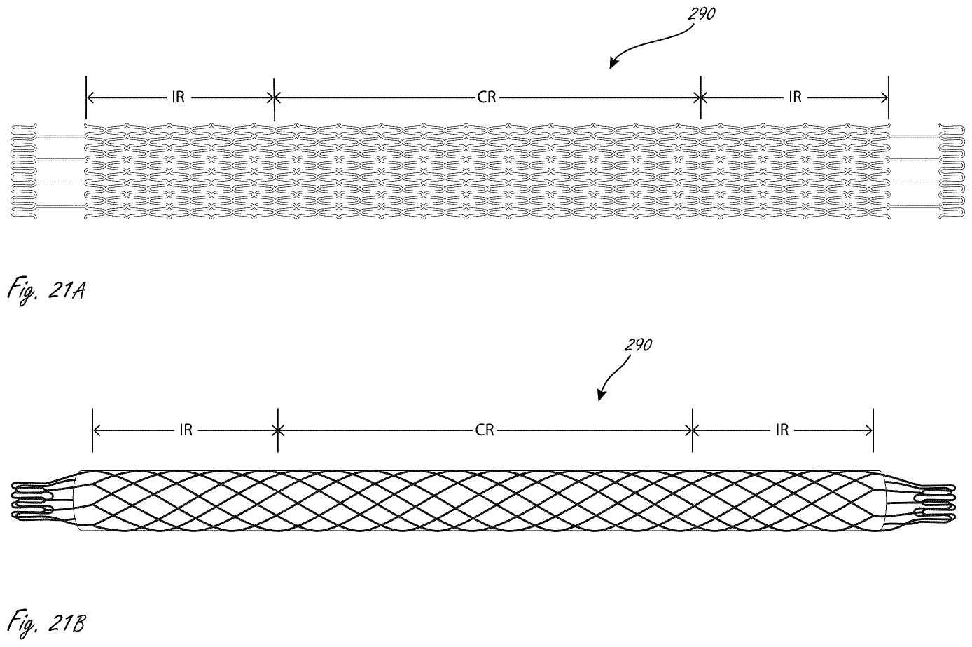

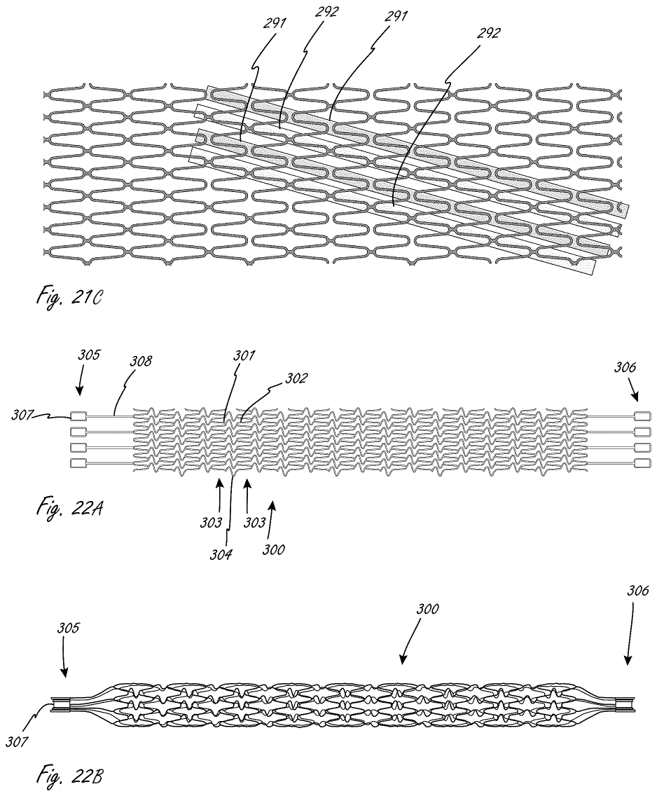

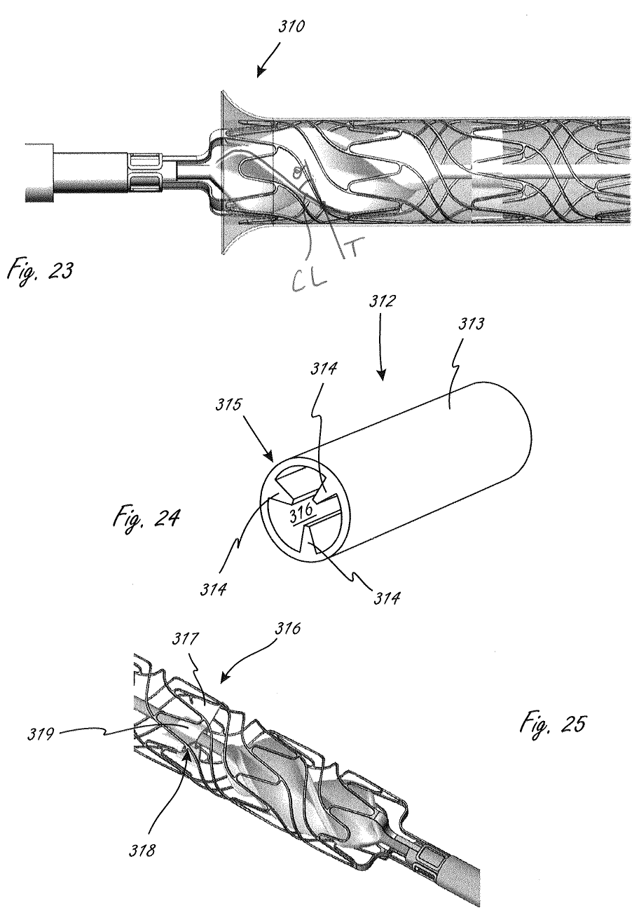

The configuration, or shape, of the plurality of helical arms can generally follow the helical shape of outermost regions of the impellers (e.g., outermost regions of helical blades) and are configured, relative to the blades, to facilitate sheathing to facilitate rotational sheathing and radial compression. Stated alternatively, the scaffold and any given blade can have strut patterns (and in particular helical arm configurations) and camber lines (a segment of one is labeled “CL” in FIG. 23), respectively, that when sheathed and unsheathed, can twist each component in a complimentary manner to perform one or more of reducing sheathing force, enhancing packing efficiency, and reducing component strains. The camber lines of the blades can generally follow the helical configuration of at least one of the helical arms in the helical regions, which can be seen in FIG. 17E, and wherein a segment of a camber line CL for one blade is shown in FIG. 23. In a side view of the pump portion (e.g., FIG. 17E or FIG. 23), and in some embodiments, a helical element and a blade can overlap at one or more locations, and a tangent “T” of the helical element and the blade camber line at the overlap location (see FIG. 23) can form an angle of 45 degrees or less, 35 degrees or less, 20 degrees or less, 15 degrees or less, or even 10 degrees or less.

There may be any number of helical regions 213 axially spaced along the support structure. Adjacent helical regions need not be equally spaced apart along the entire length of the scaffold.

In some exemplary methods of sheathing, the method may optionally include a collapsing process that includes a rotational movement of the component to which the support structure is coupled, which can be controlled by an actuator disposed outside the patient (e.g., on a handle).

In some embodiments the four proximal arms (generally labeled 218) can be inverted, such that the bend at the larger diameter portion is at a location that is further proximally than the bend at the smaller diameter portion.

The design of the scaffold in the embodiment in FIGS. 17A-17F provides a number of advantages compared to other scaffold designs. For example, the staggered proximal valleys 221 (which can also be present on the distal end of the scaffold) reduces the packing volume of the scaffold at the location of the staggered peaks. Additionally, when bent, the scaffold resists kinking and maintains a smooth curve in the bent section. This can be advantageous when placed at a target location which requires the scaffold to assume a bent configuration, such as when the scaffold is placed and extends from an ascending aorta to a left ventricle. Additionally, the design is a closed cell design (there aren't any free ends in the design; every end is connected to another section) yet retains sufficiently flexibility along the length of the scaffold. This design also includes struts that are individually terminated at the hub (proximal end of the scaffold), rather than coupled to other struts. The disconnected struts at the hub improves the manufacturing process of heat treatment, membrane coating and impeller loading. The advantages of the helical connecting members is set forth elsewhere herein.

While the scaffold design in FIGS. 17A-17F provides at least the exemplary advantages set forth herein, other scaffold designs are contemplated, and while some possible drawbacks of these alternative designs are discussed below, they (or aspects thereof) may still be used in pump portions herein. For example, depending on a particular application, one or more features may be less important than others.

FIGS. 18A-B illustrate an expandable member 250 that is one of at least two expandable members (which may also be referred herein as collapsible housings), such as the expandable members in FIGS. 3A-3D, wherein each expandable member surrounds an impeller. The scaffold design in FIGS. 18A and B has more proximal struts 251 (only one labeled) than the design in FIGS. 17A-17E (in this exemplary embodiment there are nine compared to four). Having a separate expandable member 250 for each impeller provides for the ability to have very different geometries for any of the individual impellers. Additionally, this design reduces the amount of scaffold material (e.g., Nitinol) over the length of the scaffold (compared to other full length scaffolds herein), which may offer increased tracking when sheathed). A potential challenge with this design may include creating a continuous membrane between the expandable members in the absence of an axially extending scaffolding material (see FIG. 3A). Additionally, a relatively higher number of proximal struts 251 in the outflow path may disrupt the outflow more than designs with fewer numbers of struts, such as the four struts in the embodiment in FIGS. 17A-F. Any other aspect of the expandable member(s) herein, such as those described in FIGS. 3A-3D, may be incorporated by reference into this exemplary design. FIG. 18B shows a planar view of the scaffold in a non-expanded configuration to further illustrate the design.

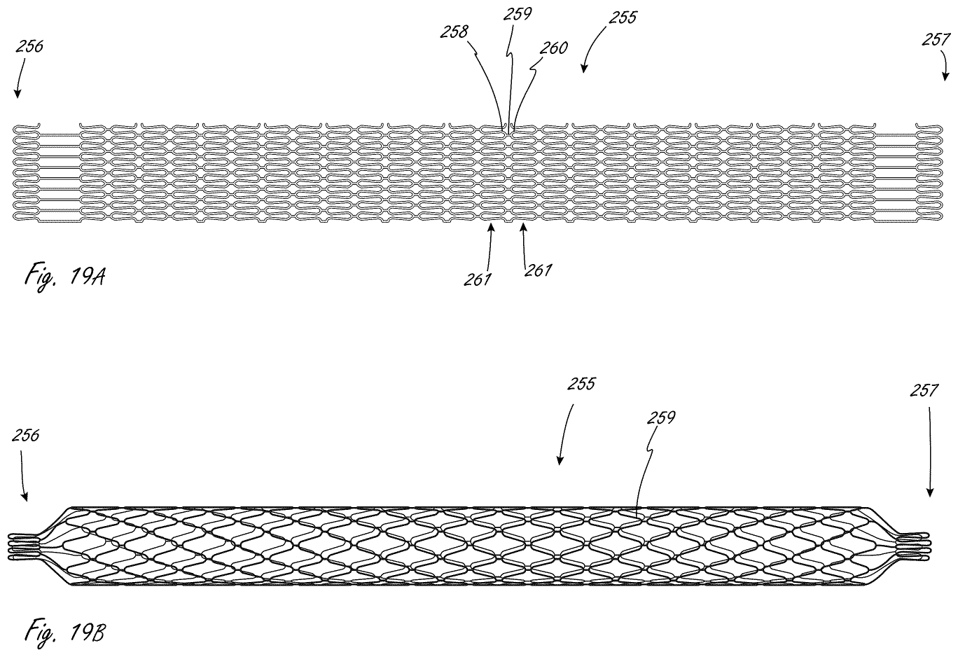

FIGS. 19A and 19B illustrates a scaffold design that has the same general pattern as in FIGS. 18A and B, but the scaffold pattern is not separated into two discrete sections, but rather the scaffold is a single elongate member as shown. FIG. 19A is a planar view of the unexpanded scaffold, while FIG. 19B is an expanded configuration. The scaffold design in FIG. 19A and B has proximal end 256 and distal end 257 (i.e., the hub coupling regions) that have continuous, integral formations, rather than the independent proximal hub ends as in the design in FIGS. 17A-F. It may be easier to apply (e.g., coat) a membrane to the single scaffold in this design and the design in FIGS. 17A-F (compared to, for example, the separate axially spaced expandable members such as in FIGS. 18A-B). An exemplary drawback may be the relatively higher number of proximal struts (nine in this embodiment), which like the design in FIGS. 18A-B may disrupt the outflow as the blood exits the fluid lumen. This particular pattern may also be too rigid for some applications or access routes where more increased bending and flexing are desired. This design is relatively rigid over the axial length, and does not bend or flex with great ease. In this design, each peak 258 and valley 260 in adjacent sections 261 are radially aligned and are coupled by connector 259, which is parallel with a longitudinal axis of the fluid lumen.

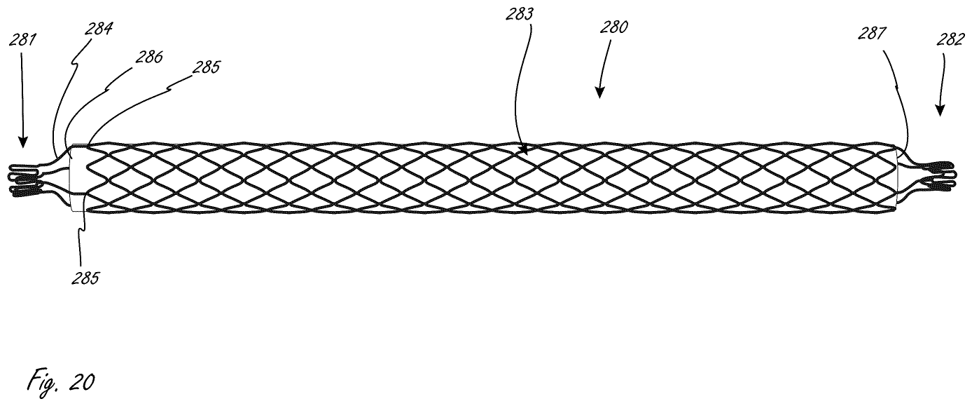

FIG. 20 illustrates an exemplary scaffold 280 that extends along the entire axial length from the proximal hub end 281 to a distal hub end 282, and wherein the hub regions have the same design as in FIGS. 19A and B. The cells 383 (only one labeled, which have a diamond pattern in the expanded configuration) in this design have a reduced size compared to the designs in FIGS. 18 and 19. The number of struts 284 in this design is also less than that in FIGS. 18 and 19 (e.g., four at each end in this design, compared to nine), which in this embodiment means that every other peak 285 (only two are labeled) at the scaffold ends is coupled to (e.g., integral to) a proximal strut 284, rather than every peak. The length of the struts in this embodiment is greater than in FIGS. 18 and 19, which enables the membrane proximal end 286 (termination location) to be axially spaced by a short distance from the ends of the terminating peaks 285, as shown in the figures. This added strut length thus allows the membrane (or conduit) to be introduced into the sheath prior to the terminating peaks, which can reduce the likelihood of the proximal peaks from catching on the sheath during the sheathing process. This design, like other full length scaffolds, makes it easier to apply a membrane to the scaffold along its length. Peaks and valleys in adjacent sections are each coupled with a short linear connector. This design, like that in FIGS. 19A and B, is relatively rigid over its length and does not have particularly strong bending or flexing characteristics, which may be required for some applications. Additional potential drawbacks to this design include inadequate compressive resistance during unsheathing, difficult sheathing due to the geometry/pattern of the scaffold; and the expandable hub sections at the proximal and distal ends could become sensitive to fatigue and plastic deformation.

FIGS. 21A-C show a scaffold design that is similar to FIG. 20A, but with differences described below. In a central region “CR”, between which proximal and distal impellers would be located, the design attempts to improve on the flexibility compared to FIGS. 20A and B. The benefits of this increased flexibility in this region are described herein. In the central region, alternating cell connections are removed, as shown, to improve flexibility in this region. In each axial section, alternating (radially) peaks are coupled, and alternating valleys are coupled. These removed alternating cell connections create helical regions 291 (see FIG. 21C) around the scaffold which do not include connection elements and helical regions 292 around the scaffold which include connection elements (see the magnified view in FIG. 21C). The helical regions alternative between no-connections regions 291 and connection regions 292.

Central region “CR” flexibility is increased in this design compared to the design in FIG. 20-B because of the no-connection regions 291, and the scaffold has relatively more rigid impeller sections “IR” adjacent the central region where the impellers are disposed (not shown). The relatively increased rigidity in the impeller regions IR can help maintain tip gap and impeller concentricity. This scaffold pattern thus provides for a flexibility distribution, along its length, of a proximal section of relatively less flexibility (“IR”), a central region “CR” of relatively higher flexibility, and a distal section “IR” of relatively less flexibility. The relatively less flexibility sections (i.e., the two IR regions) are where proximal and distal impellers can be disposed (not shown but other embodiments are fully incorporated herein in this regard), with a relatively more flexible region in between. The benefits of the relative flexibility in these respective section are described elsewhere herein.

In this design, the lack of alternating connectors in the central region CR may, however, make the shape set and membrane application process more difficult, however. The uncoupled (i.e., not connected) regions of the scaffold in the central region may also rub against and cut into the membrane, increasing the likelihood of membrane failure at those locations. Additionally, flexibility of the scaffold along its length may still not be adequate once membrane was applied, depending on the applications, the target placement location within the patient, access route. Additional possible drawbacks based on similar features are set forth above with reference to FIG. 20 (e.g., sheathing difficult due to geometry of scaffold).

FIGS. 22A and 22B illustrate a scaffold design 300 that is similar to FIGS. 21A and B, but with differences described below. Any feature from scaffolds herein can be incorporated into this scaffold design. The design includes peaks 301 and valleys 302 (only one set labeled for clarity) on axially adjacent sections 303 that are connected with spring connectors 304 (only one labeled for clarity) that couple radially-aligned peaks 301 and valleys 302 on adjacent sections. The spring-like connectors 304 in this design provide better flexibility along the length of the scaffold compared to FIGS. 21A-B. This is partially because the spring connectors 304 provide individual cell articulation.

The proximal and distal hub ends 305 and 306, respectively, have four independent, free-ended (i.e., not coupled to each other) members 307 (only one labeled at the proximal end for clarity) that are coupled to respective hubs (not shown). The struts 308 (only one labeled) thus have more flexibility relative to one another. More or fewer members 307 may be present at end each (e.g., two members at each end), and the ends may have different numbers of members (e.g., four at the proximal end, eight at the distal end). The individual (i.e., disconnected) members 307 at the hub ends improved manufacturing process of heat treatment, membrane coating and impeller loading. Some potential drawbacks for this design, depending on the particular application, may be that during unsheathing, strut buckling may occur due to inadequate compressive resistance. Additionally, the sheathing force may be undesirably high, which may be due at least partially to the membrane. Additionally, flexibility may be deemed inadequate once the membrane is applied to the scaffold.

For a particular exemplary application in which the pump portion is navigated for placement across an aortic valve (aspects of which are described herein), the scaffold design in FIG. 17A-F can provide the benefits set forth herein, whereas the scaffolds in FIGS. 18-22 may be suboptimal in one or more regards for this particular application (e.g., not sufficiently flexible, membrane application to scaffold suboptimal process, etc.). For some applications, however, one or more features of the scaffolds in FIGS. 18-22 may be desired. For example, the blood pump may be placed in a location where flexibility is not as important, or where relatively high stiffness across the length of the scaffold is desired or tolerated. Any of the features in the scaffold designs in FIGS. 18-22 can thus be combined in any suitable combination to provide a scaffold structure that provide desired functionality. For example, the design in FIGS. 17A-F could instead have hub regions that are not distinct members (like in FIGS. 22A-B), but are instead continuous like the designs in FIGS. 19-21.

FIG. 23 illustrates a proximal region of an exemplary pump portion, features of which can be incorporated into any of the pump portions herein. Not all of the features in the embodiment in FIG. 23 need to be included in the pump portion shown. The proximal end 310 of the fluid lumen has a flared radially outward configuration as shown with a smooth curve (the end of the lumen is furthest radially outward), which can facilitate radial flow of the impeller at the outflow (which is optionally a proximal impeller, and optionally one of a plurality of impellers). The distal end of the fluid lumen may also have the same or similar type of flared configuration (not shown), with or without a flared proximal end. A flared distal end configuration may limit the amount of contact between more rigid parts of the pump portion and a left ventricle wall (if that is where it is placed), reduce the likelihood of tissue being pulled into contact with the rotating impellers, it can prevent or minimize blockage of the pump portion inflow, and it may help prevent migration of the pump portion by acting as an enlarged interface region that can interface with native tissue and prevent further migration (e.g., engaging native valve tissue like leaflet and preventing the pump portion from passing through the valve opening). Any other suitable aspect of this disclosure is incorporated by reference into this embodiment.

Some aspects of the disclosure herein describe a pump portion that includes one or more central members (which may be referred to as “intermediate members” herein), optionally stationary and optionally between two first and second impellers (e.g., see FIGS. 10-13C). FIG. 24 is a perspective view and illustrates a portion of an exemplary conduit (optionally collapsible) that has a central member incorporated therein. Incorporated herein in this context includes a central member that is integrally formed with the conduit, as well as a central member that is attached to a collapsible conduit such that it is considered part of the collapsible blood conduit. For example, the central member could have an outer surface 313 that is attached (e.g., bonded) to an inner surface of any of the collapsible conduits herein (e.g., attached to a flexible membrane portion of the conduit), such that it is considered part of the collapsible conduit. The central member shown in FIG. 24 can have one or more flow modifying elements (e.g., blades) 314 extending radially inward from a peripheral portion 315 towards a central region 316 (not extending from a central region), but which are not coupled together at a central hub. The flow modifying elements 314 (e.g., blades) in FIG. 24 (or any other embodiment or claim herein), being part of an object that is not in rotational operation with an impeller, may be referred to herein as part of a stator, or diffuser vanes. The flow modifying elements can each have a variety of cross sectional geometries. The flow modifying elements can be configured to increase fluid pressure between impellers (with a drop in velocity) and/or reduce swirl velocity at its location, optionally between distal and proximal impellers.

In some embodiments the central member can have a plurality of blade like extensions that are chords of the peripheral curved portion (which may have a circular cross section), such that the chords do not have free ends as to do the flow modifying elements 314 in FIG. 24. One or more chords can extend from and to different regions of the peripheral curved portion. If there are a plurality of chords, and in an end sectional view, the chords can have different lengths between the two end points where they couple to (integrally or attached to) the outer peripheral region.

FIG. 25 is a perspective view of a proximal portion of a pump portion including an expandable scaffold and at least one impeller (not labeled but are easily identifiable based on other figures herein). The pump portion also includes intermediate member 316, which in some ways is similar to other intermediate, or central, members herein. Intermediate member 316 includes flow modifying elements 317 (e.g., blades), each of which has an outermost engagement feature 318 (e.g., a flange) that is configured to stably interface with a corresponding mating feature (e.g., a peak or a valley) in the scaffold. The blade/scaffold engagement can cause collapse of the blades during radial compression and sheathing of the scaffold. Additionally, the blades and scaffold can interface or be coupled using a variety of techniques. For example, the flow modifying elements and scaffold can be coupled by spot welding. In some embodiments a flow modifying element 317 end can have a feature that interlocks with a feature on a central hub 319 (e.g., a dovetail interlocking relationship), with the locating features on central span bearing(s), not a drive cable. This may make manufacturing easier. The outer housing in this embodiment can be any of the outer housings herein.

One or more impellers that are part of a blood pump system (such as any herein) may be rotated at relatively high speeds, such as between 10,000 and 50,000 RPM. Impellers can be rotated by being in rotational communication with a drive member (e.g., a drive cable) or other component in rotational communication with the impeller, which can be rotated by an energy source (e.g., motor). Rotating the drive member at the same RPMs as the impellers may cause wear on the drive member, vibration, and perhaps requires lubricating (aspects of exemplary lubricating systems are described elsewhere herein) the drive member. It may be advantageous to have the drive member rotating at speeds less than the impellers, while still causing the impellers to rotate at the desired higher RPMs. One aspect of this disclosure is a blood pump that includes one or more drive members that can be rotated at lower RPMs than one or more impellers. This may decrease drive member wear, reduce lubrication needs, and reduce vibration. This may be particularly advantageous in applications in which the blood pumps are used for relatively long terms (e.g., 24 hours or more). For example, this may be particularly advantageous for cardiogenic shock indications.

The rotating drive member (e.g., drive cable, magnetic stator) can rotate slower than the one or more impellers. In some exemplary embodiments the rotating drive member may be rotating between zero and one times (1×) the impeller RPM. For example, if any impeller is rotating at 20,000 RPM, the drive member may be rotating between zero and 20,000 RPM. In some embodiments the drive member may be rotating between 0.25 and 1× the impeller RPM, or between 0.3 and 1×, or between 0.4 and 1×, or between 0.5 and 1×, or between 0.6 and 1×, or between 0.7 and 1×, or between 0.8 and 1×, or between 0.9 and 1×.

FIG. 26A illustrates only portions of an exemplary blood pump to illustrate an exemplary embodiment of how to rotate an impeller faster than a drive member. The exemplary speed increase mechanism in FIG. 26A utilizes gearing to accomplish the speed increase, with an output gear with a smaller diameter than the input gear, causing more speed at the output gear axis, about which the output shaft rotates. The output shaft (and the impeller to which it is coupled) thus rotates faster than the input shaft (e.g., drive member). FIG. 26B illustrates how to use multiplicative gearing to get a greater difference (compared to FIG. 26A) in speeds between the input shaft (e.g., drive member) and output shaft 2 (to which the impeller is coupled). Input gear 2 has a greater diameter than output gear 2.

Additionally gearing systems such as planetary gear boxes and magnetic gear boxes can also be used to increase the speed the one or more impellers relative to the rotation of the input drive member.

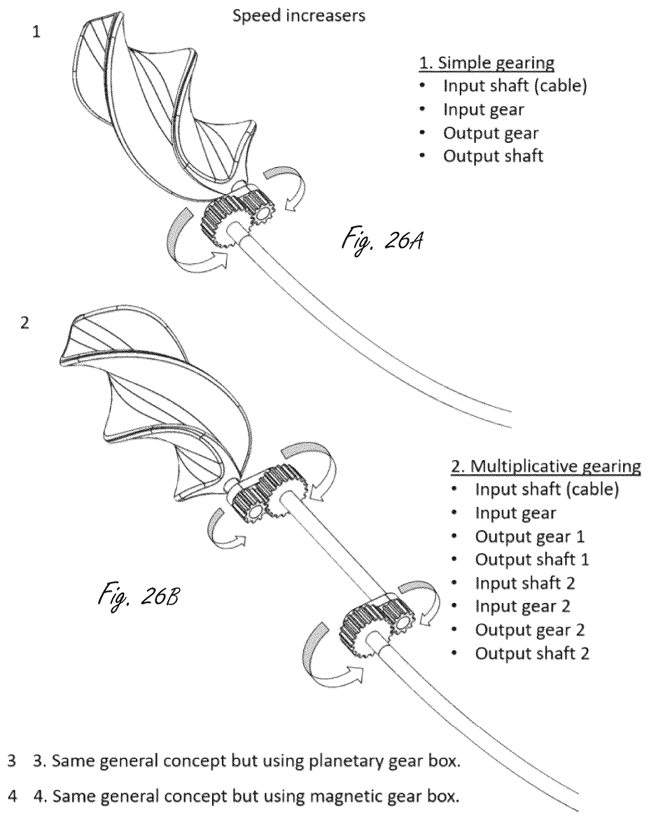

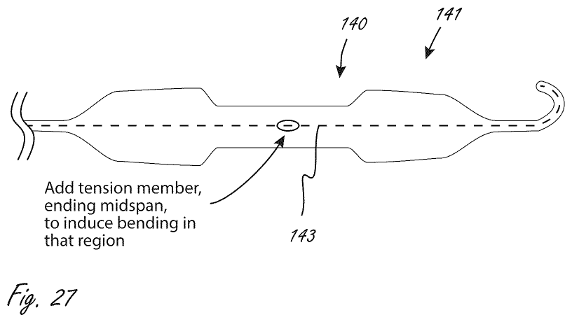

FIG. 27 illustrates an exemplary design of a pump portion 140 that includes at least one tensioning member 143 (e.g., pullwire) that, when tensioned, induces bending in at least a portion of the pump portion. A tensioning member may extend as far distally as in between distal and proximal impellers (or further distally or further proximally), and can cause a bend to form between the two impellers after the pump portion is deployed from a delivery system. For example, a handle can include an actuator (lever, button, etc.) that when actuated tensions the one or more tensioning members, causing deflection in region. All known tensioning member (e.g., pullwire) designs and uses can be incorporated into this embodiment to implement the one or deflectable regions. For example the catheter can include one or more pullwire lumens extending along any portion thereof, wherein the pullwire(s) is adhered at its distal end to one or more parts of the catheter depending on the location(s) of the desired deflection region.

The following disclosure provides exemplary method steps that may be performed when using any of the blood pumps, or portions thereof, described herein. It is understood that not all of the steps need to be performed, but rather the steps are intended to be an illustrative procedure. It is also intended that, if suitable, in some instances the order of one or more steps may be different.

Before use, the blood pump can be prepared for use by priming the lumens (including any annular spaces) and pump assembly with sterile solution (e.g., heparinized saline) to remove any air bubbles from any fluid lines. The catheter, including any number of purge lines, may then be connected to a console. Alternatively, the catheter may be connected to a console and/or a separate pump that are used to prime the catheter to remove air bubbles.

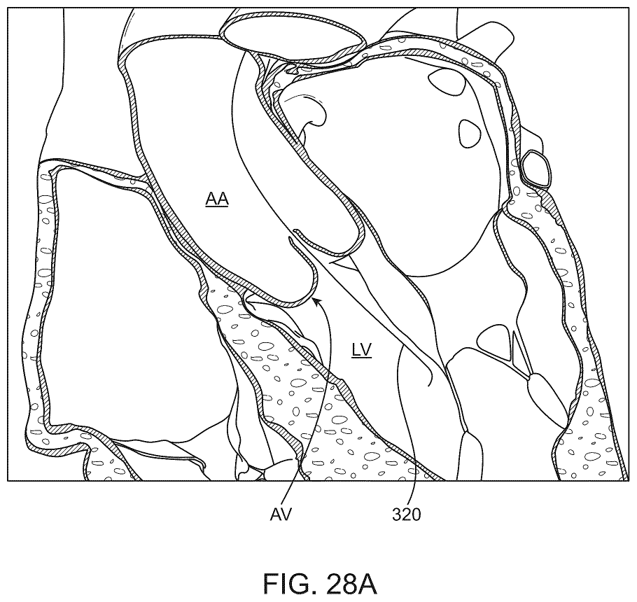

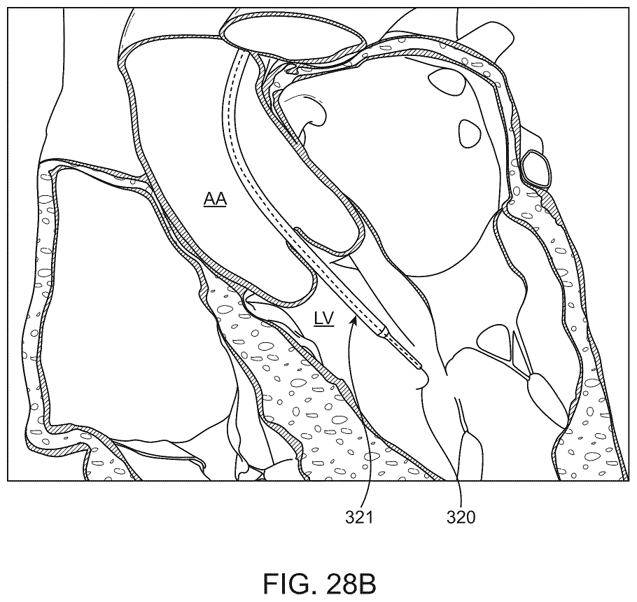

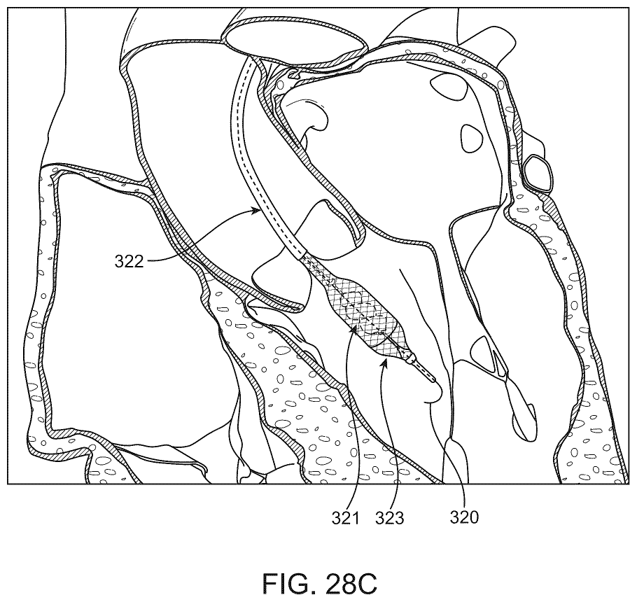

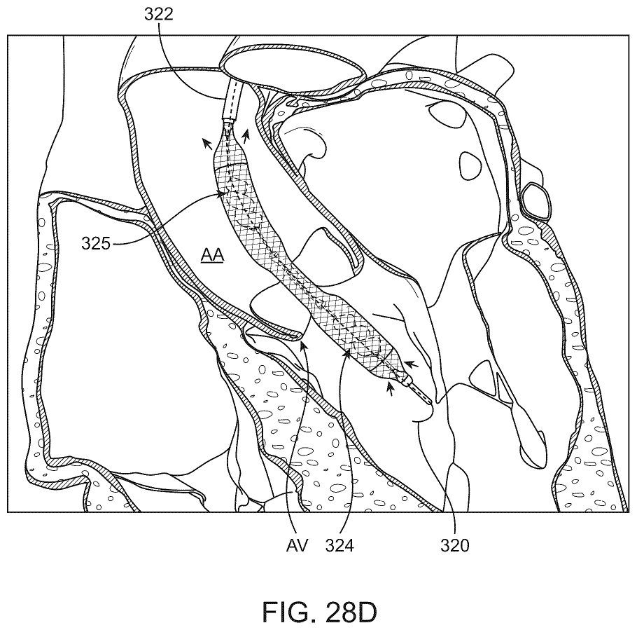

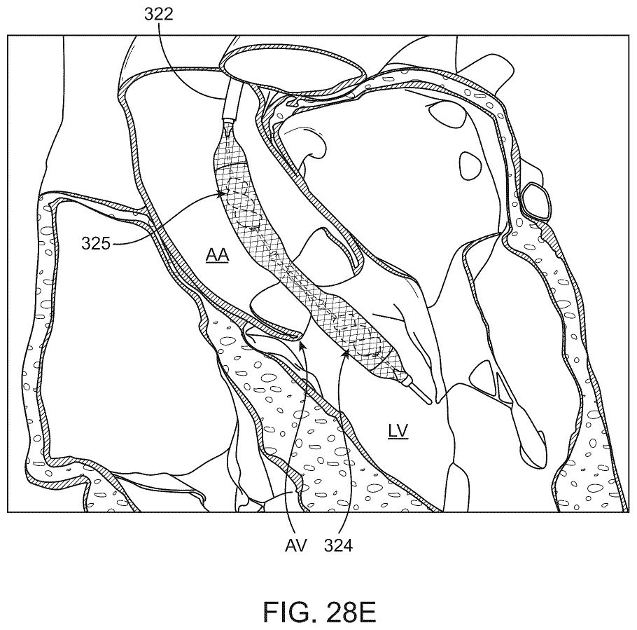

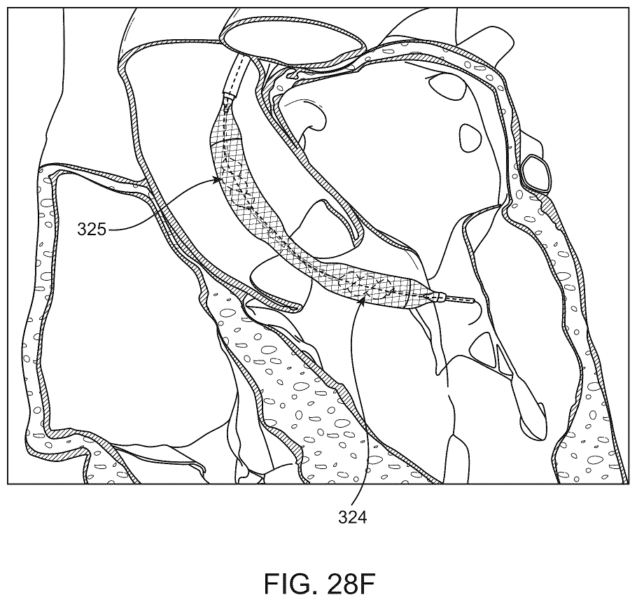

After priming the catheter, access to the patient's vasculature can be obtained (e.g., without limitation, via femoral access) using an appropriately sized introducer sheath. Using standard valve crossing techniques, a diagnostic pigtail catheter may then be advanced over a, for example, 0.035″ guide wire until the pigtail catheter is positioned securely in the target location (e.g., left ventricle). The guidewire can then be removed and a second wire 320 (e.g., a 0.018″ wire) can be inserted through the pigtail catheter. The pigtail catheter can then be removed (see FIG. 28A), and the blood pump 321 (including a catheter, catheter sheath, and pump portion within the sheath; see FIG. 28B) can be advanced over the second wire towards a target location, such as spanning an aortic valve “AV,” and into a target location (e.g., left ventricle “LV”), using, for example, one or more radiopaque markers to position the blood pump.

Once proper placement is confirmed, the catheter sheath 322 (see FIG. 28C) can be retracted, exposing first a distal region of the pump portion. In FIG. 28C a distal region of an expandable housing has been released from sheath 322 and is expanded, as is distal impeller 324. A proximal end of housing 323 and a proximal impeller are not yet released from sheath 322. Continued retraction of sheath 322 beyond the proximal end of housing 323 allows the housing 323 and proximal impeller 325 to expand (see FIG. 28D). The inflow region (shown with arrows even though the impellers are not yet rotating) and the distal impeller are in the left ventricle. The outflow (shown with arrows even though the impellers are not rotating yet) and proximal impeller are in the ascending aorta AA. The region of the outer housing in between the two impellers, which may be more flexible than the housing regions surrounding the impellers, as described in more detail herein, spans the aortic valve AV. In an exemplary operating position as shown, an inlet portion of the pump portion will be distal to the aortic valve, in the left ventricle, and an outlet of the pump portion will be proximal to the aortic valve, in the ascending aorta (“AA”).

The second wire (e.g., an 0.018″ guidewire) may then be moved prior to operation of the pump assembly (see FIG. 28E). If desired or needed, the pump portion can be deflected (active or passively) at one or more locations as described herein, as illustrated in FIG. 28F. For example, a region between two impellers can be deflected by tensioning a tensioning member that extends to a location between two impellers. The deflection may be desired or needed to accommodate the specific anatomy. As needed, the pump portion can be repositioned to achieve the intended placement, such as, for example, having a first impeller on one side of a heart valve and a second impeller on a second side of the heart valve. An exemplary pump portion with an exemplary tensioning member is shown in FIG. 27. It is understood that in FIG. 28F, the pump portion is not in any way interfering or interacting with the mitral valve, even if it may appear that way from the figure.

Any number of purge lines may then be attached to the proximal portion of the blood pump that is disposed outside of the patient. For example, fluid inlet(s) lines and fluid outlet(s) lines may be attached to one or more fluid ports on the proximal portion of the blood pump. A purge process can then be initiated to move fluid into the blood pump through at least one fluid pathway. One or more Confirmation steps can be performed to confirm the purge is operating as intended before turning on the pump. The pump assembly can then be operated, causing rotation of the one or more impellers. Any one of flow rate(s), pressure(s), and motor operation can be monitored at any time.

FIGS. 29-35B illustrate additional exemplary intermediate members, which may function at least partially as stators (and may also provide radial support), and that include a plurality of fluid modifiers that may be disposed between distal and proximal impellers, and are adapted to influence blood flow between the impellers, other examples of which are provided herein. Any aspect of FIGS. 29-35B may be incorporated with any other aspect of the blood pumps herein. For example, impellers might not be shown in FIGS. 29-35B for clarity but it is understood that one or more impellers can be incorporated into these embodiments.

FIGS. 29A and 29B illustrate an exemplary blood conduit 402 with flow modifiers 402 extending radially inward from an inner surface of the conduit. The flow modifiers (e.g., blades) do not extend to a central hub, but rather they have free radially inner ends, which can be seen more clearly in the end view of FIG. 29B. The flow modifiers 402 (e.g., stator elements) may be molded individually, then secured to the inner surface of the conduit (e.g., to a membrane).

Conduit 402 may also include any other supporting members herein, such as, without limitation, any of the nitinol scaffolds herein. Flow modifiers may have slight curvature to them, which can be seen in FIG. 29B. Flow modifiers 402 may be considered part of one stator.

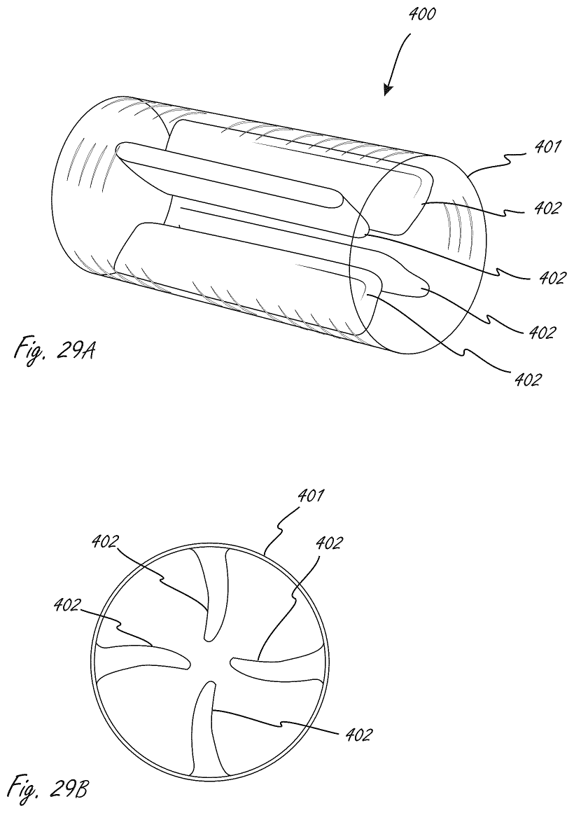

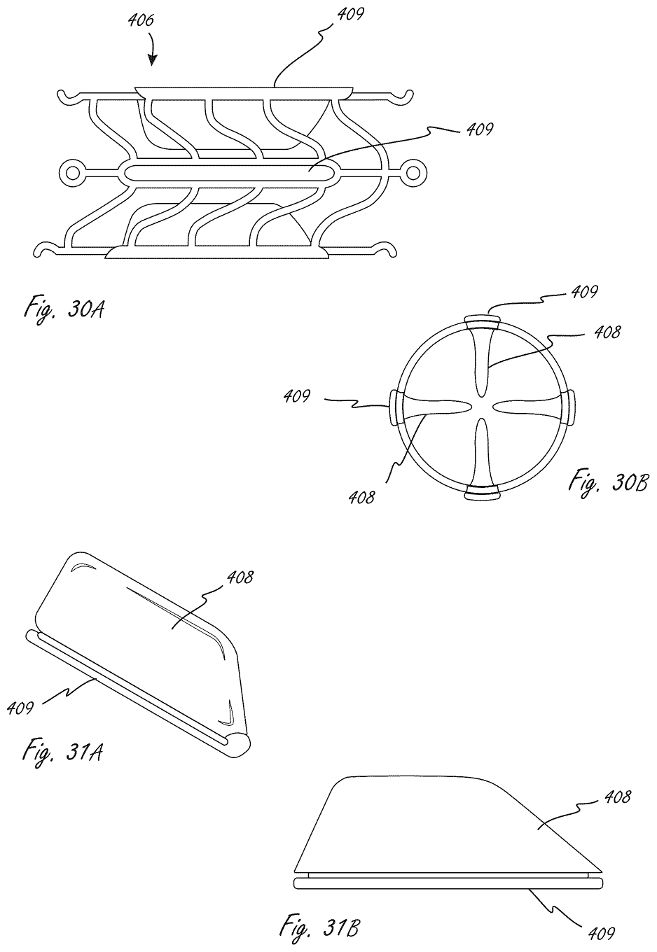

FIGS. 30A, 30B, 31A and 31B illustrate an exemplary support member that has a plurality of apertures therein, each configured to receive therethrough and interface with a flow modifier 408, which have enlarged regions 409 that interface with a part of the support member to help stabilize the position of the flow modifier relative to the support member. The location of elongate apertures 407 establish the position and orientation of the flow modifiers (e.g, stator elements). After the flow modifiers are inserted into the apertures, a conduit material (e.g, membrane) may be sprayed over the subassembly to further secure the modifiers 408 in place. Alternatively, the material could be sprayed first, then a cut made through the apertures 407, into which the modifiers may be inserted. An additional layer of material could be sprayed to create a seal. Flow modifiers 408 may be considered part of one stator.

FIGS. 32A-32C illustrate an exemplary support member 420 (e.g., scaffold, eg nitinol) in which one or more flow modifiers 421 are integrated (integral) into the expandable scaffold structure, which can be directed radially inward in the shape set process, thus creating a flow modifier (e.g, stator. A material (eg polymer) can be applied to the member 420 to create a blood conduit. The scaffold and flow modifiers could also be designed to be fabricated separately and interlocked after a membrane material (e.g. polymer) is applied to the scaffold. The scaffold can have any pattern described herein. Flow modifiers 421 may be considered part of one stator.

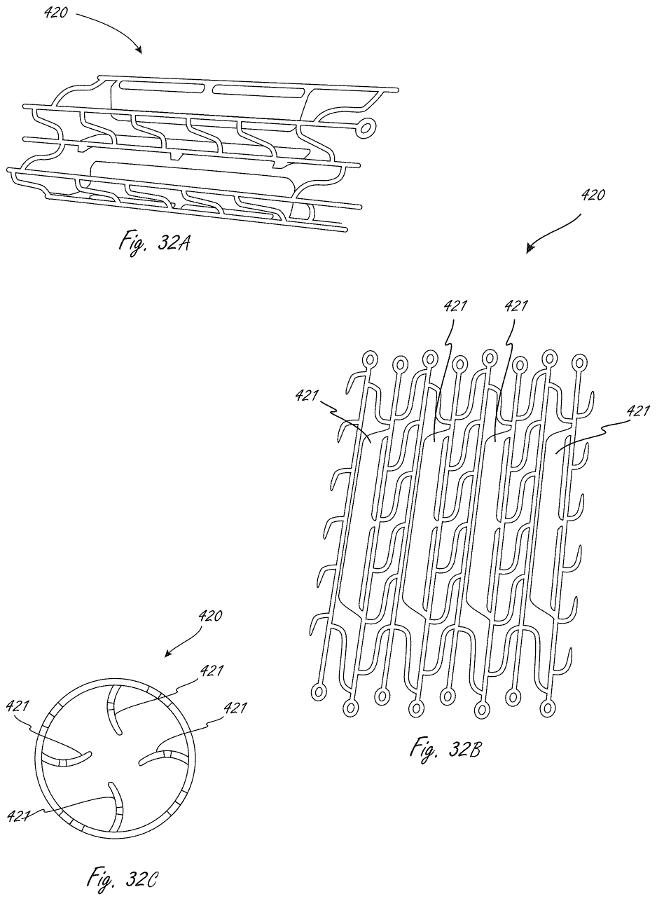

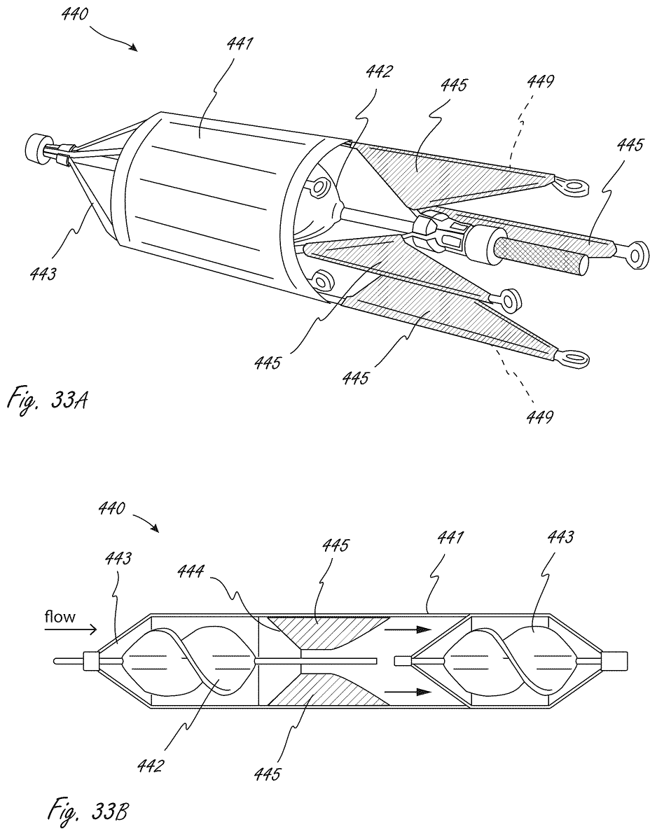

FIGS. 33A and 33B illustrate an exemplary collapsible pump portion 440 including conduit 441, distal impeller 442, and proximal impeller 443. The pump also includes a plurality of flow modifiers 445, which are secured to struts 444 in the pump. The struts can be part of a distal impeller basket, examples of which are described herein. In this embodiment the flow modifier are secured to proximal struts of a distal impeller basket, but could be secured to distal or proximal struts of a proximal impeller basket. Flow modifiers in this embodiment can be flexible membrane, or other relatively flexible and thin material. The flow modifiers are positioned to manage flow over the strut, as well as being fixed to a longitudinal spine element 449 (lines shown in phantom since the element may be covered by flow modifier material 445) extending from a scaffold to aid in directing flow longitudinally through the blood conduit. The flow modifiers (eg stators) could be flexible polymer with or without fabric reinforcement to aid in collapsibility and sheathing. Flow modifiers 445 may be considered part of one stator.

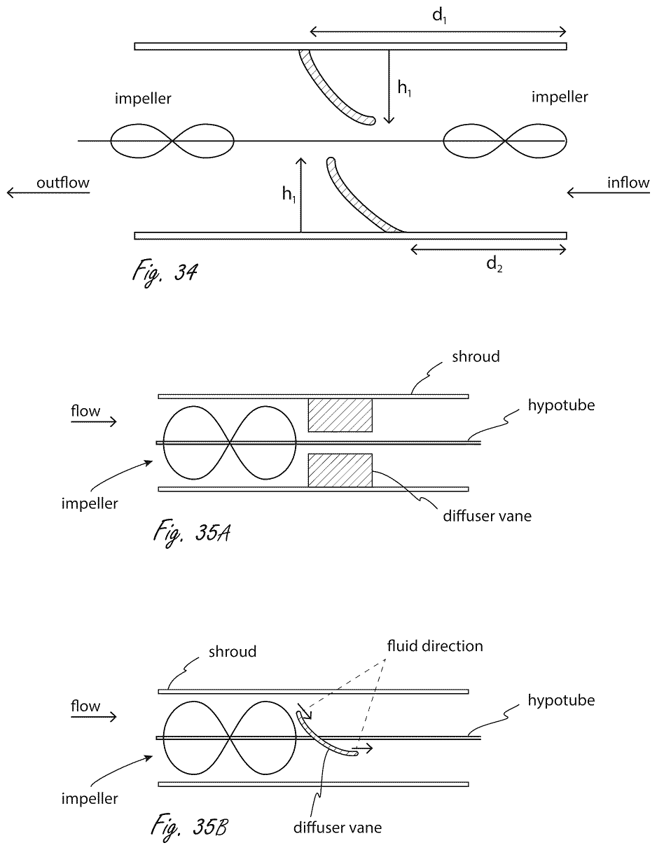

The disclosure that follows, including FIGS. 34, 35A and 35B, may be referred to as hugging collapsible diffusers to increase outflow pressure. FIG. 34 illustrates a concept with hugging vanes of diffuser/stator. FIGS. 35A and 35B illustrates the profile of hugging vanes (A) side view (B) top view. Diffusers are generally designed as stationary components to convert rotational speed/energy of flow into desired additional pressure by de-swirling the flow (i.e. removing the rotational velocity component of fluid). The shape of a diffuser (stator) plays an important role in efficiency of this process. Pump portions here in are generally collapsible for delivery (eg to an aortic valve), and then are expanded for use.

FIGS. 34-35B illustrate an exemplary concept is which vanes of the diffuser have a special geometry/configuration that hug/spoon each other to allow crimping of the pump to the desired delivery profile size. The concepts elsewhere herein related to flow modifiers can be incorporated into these embodiments as well (e.g, scaffolds interfacing with flow modifiers).

With respect to FIG. 34, the diffusers can be designed to be conformable, part of the shroud/blood conduit and adapted to increase pressure further. In some embodiments, an elastic material like nitinol can be used for an inner part of the diffusers, which can be coated with a material (e.g., polymer, which may be the same material as the blood conduit material) to create an diffuser outer surface.

Any of the stators herein, including any of the flow modifying elements (aka flow modifiers), can be incorporated with any suitable aspect of any shroud, housing, blood flow conduit, impeller basket, etc., that is described herein, including any methods of manufacturing the same.

In some embodiments the diffusers can be made of the same or similar material to the blood conduit membrane. Injection molding can be used to make the diffusers.

An exemplary clinical advantage e could be to maintain RPM of the drive cable/pump within reasonable limit to avoid hemolysis, while still increasing pressure to a desired range using a pump with the diffusers.

What is claimed is:

1. An intravascular blood pump, comprising:
a pump portion that includes:
a collapsible blood conduit defining a blood flow lumen between an inflow and an outflow;
a proximal collapsible impeller axially spaced from a distal collapsible impeller, at least a portion of each of the distal and proximal collapsible impellers disposed between the inflow and the outflow; and
a proximal collapsible basket in which the proximal impeller is disposed, the proximal collapsible basket providing radial support to the blood conduit at the location of the proximal impeller;
a distal collapsible basket in which the distal impeller is disposed, the distal collapsible basket providing radial support to the blood conduit at the location of the distal impeller;
a collapsible radial support member supporting one or more of a distal region of the proximal collapsible basket, a proximal region of the distal collapsible basket, or a central section of the blood conduit disposed axially between the proximal basket and the distal basket.

2. The blood pump of claim 1, wherein the radial support member includes an annular peripheral member and a plurality of support elements extending radially inward from the annular peripheral member.

3. The blood pump of claim 2, wherein the plurality of support elements do not extend to a central hub.

4. The blood pump of claim 2, wherein the plurality of support elements extend to a central hub.

5. The blood pump of claim 2, wherein the plurality of support elements have radially inner free ends.

6. The blood pump of claim 2, wherein the radial support member supports the distal region of the proximal basket.

7. The blood pump of claim 2, further comprising a second radial support member spaced axially from the radial support member, the second radial support member is positioned to radially support the proximal region of the distal basket.

8. The blood pump of claim 7, wherein the second radial support member includes a second annular peripheral member and a plurality of second support elements extending radially inward from the second annular peripheral member.

9. The blood pump of claim 1, wherein the radial support member supports the proximal region of the distal basket.

10. The blood pump of claim 1, wherein the collapsible radial support member comprises a stator including one or more blood modifying elements, such as any of the blood modifying elements herein.

11. An intravascular blood pump, comprising:
a pump portion that includes:
a collapsible blood conduit defining a blood flow lumen between an inflow and an outflow;
a proximal collapsible impeller axially spaced from a distal collapsible impeller, at least a portion of each of the distal and proximal collapsible impellers disposed between the inflow and the outflow; and
a proximal collapsible basket in which the proximal impeller is disposed, the proximal collapsible basket providing radial support to the blood conduit at the location of the proximal impeller;
a distal collapsible basket in which the distal impeller is disposed, the distal collapsible basket providing radial support to the blood conduit at the location of the distal impeller;
a collapsible radial support member including an annular peripheral member and a plurality of support elements extending radially inward from the annular peripheral member,
the collapsible radial support radially supporting one or more of a distal region of the proximal collapsible basket, a proximal region of the distal collapsible basket, or a central section of the blood conduit disposed axially between the proximal basket and the distal basket.

12. The blood pump of claim 11, wherein the plurality of support elements do not extend to a central hub.

13. The blood pump of claim 11, wherein the plurality of support elements extend to a central hub.

14. The blood pump of claim 13, wherein the plurality of support elements have radially inner free ends.

15. The blood pump of claim 13, wherein the collapsible radial support is disposed radially within at least one of the distal region of the proximal collapsible basket, a proximal region of the distal collapsible basket.

16. The blood pump of claim 11, wherein the collapsible radial support is disposed radially within the distal region of the proximal basket, wherein the proximal basket includes a plurality of proximal struts but does not include a plurality of distal struts.

17. The blood pump of claim 11, wherein the collapsible radial support comprises a stator, the stator including the plurality of support elements.

18. The blood pump of claim 17, wherein the plurality of support elements are configured to increase fluid pressure between the distal and proximal impellers.

19. A method of deploying an intravascular blood pump across an aortic valve, comprising:
advancing an intravascular blood pump to a region of a heart valve, the intravascular blood pump comprising a distal basket, a distal impeller, a proximal basket, a proximal impeller, and a blood flow conduit;
deploying the distal basket and the distal impeller each from collapsed delivery configurations to deployed configurations;
positioning at least a portion of the distal basket in a left ventricle so that a distal end of the distal basket is distal to aortic valve leaflets;
deploying the proximal basket and the proximal impeller each from collapsed delivery configurations to deployed configurations;
positioning at least a portion of the proximal basket in an ascending aorta so that a proximal end of the proximal basket is proximal to the aortic valve leaflets;
positioning a central region of the conduit that is axially in between the deployed distal basket and the deployed proximal basket adjacent the aortic valve leaflets;
activating the distal impeller to impel blood towards the proximal impeller;
modifying the flow of blood with at least one flow modification member at at least one flow modification location that is proximal to the distal impeller and distal to the proximal impeller;
activating the proximal impeller to impel blood to an outflow of the conduit.

20. The method of claim 19, wherein modifying the flow of blood with at least one flow modification member comprises increasing fluid pressure between the distal and proximal impellers.

21. A method of deploying an intravascular blood pump across an aortic valve, comprising:
advancing an intravascular blood pump to a region of a heart valve, the intravascular blood pump comprising a distal basket, a distal impeller, a proximal basket, a proximal impeller, and a blood flow conduit;
deploying the distal basket and the distal impeller each from collapsed delivery configurations to deployed configurations;
positioning at least a portion of the distal basket in a left ventricle so that a distal end of the distal basket is distal to aortic valve leaflets;
deploying the proximal basket and the proximal impeller each from collapsed delivery configurations to deployed configurations;
positioning at least a portion of the proximal basket in an ascending aorta so that a proximal end of the proximal basket is proximal to the aortic valve leaflets;
positioning a central region of the conduit that is axially in between the deployed distal basket and the deployed proximal basket adjacent the aortic valve leaflets;
activating the distal impeller to impel blood towards the proximal impeller;
activating the proximal impeller to impel blood to an outflow of the conduit;
deploying a collapsible radial support to radially support at least one of a distal region of the proximal collapsible basket, a proximal region of the distal collapsible basket, or a central section of the blood conduit disposed axially between the proximal basket and the distal basket.

22. The method of claim 21, wherein deploying the collapsible radial support comprises deploying an annular peripheral member and a plurality of support elements extending radially inward from the annular peripheral member.

23. The method of claim 22, wherein deploying the collapsible radial support comprises deploying the annular peripheral member radially within a proximal region of the distal basket.

24. The method of claim 22, wherein deploying the collapsible radial support comprises deploying the annular peripheral member radially within a distal region of proximal basket.

25. The method of claim 24, further comprising deploying a second collapsible radial support within a proximal region of the distal basket to radially support the proximal region of the distal basket.
